SEQUENCE LISTING (22 sequences)

SEQ ID NO: 1 - protein, 775 aa, Pyrococcus horikoshii
MILDADYITEDGKPIIRIFKKENGEFKVEYDRNFRPYIYALLRDDSAIDEIKKITAQRHGKVVRIVETEKIQRKFLGRPIEVWKLYLEHPQDQPAIRDKIREHPAVVDIFEYDIPFAKRYLIDKGLTPAEGNEKLTFLAVAIAALYHEGEEFGKGPVIMISYADEEGAKVITWKKIDLPYVEVVSSEREMIKRLIRVIKEKDPDVIITYNGDNFDFPYLLKRAEKLGIKLLLGRDNSEPKMQKMGDSLAVEIKGRIHFDLFPVIRRTINLPTYTLEAVYEAIFGKPKEKVYADEIAKAWETGEGLERVAKYSMEDAKVTYELGREFFPMEAQLARLVGQPVWDVSRSSTGNLVEWFLLRKAYERNELAPNKPDEKEYERRLRESYEGGYVKEPEKGLWEGIVSLDFRSAGPSIIITHNVSPDTLNREGCEEYDVAPKVGHRFCKDFPGFIPSLLGQLLEERQKIKKRMKESKDPVEKKLLDYRQRVIKILANSYYGYYGYAKARWYCKECAESVSAWGRQYIDLVRRELEARGFKVLYIDTDGLYATIPGVKDWEEVKRRALEFVDYINSKLPGVLELEYEGFYARGFFVTKKKYALIDEEGKIVTRGLEIVRRDWSEIAKETQARVLEAILKHGNVEELVKIVKDVTEKLTNYEVPPEKLVIYEQITRPINEYKAIGPHVAVAKRLMARGIKVKPGMVIGYIVLRGDGPISKRAISIEEFDPRKHKYDAEYYIENQVLPAVERILKAFGYKREDLRWQKTKQVGLGAWIKVKKS

SEQ ID NO: 2 - DNA, 59 bp, synthetic construct
  source 1..59: mol_type = other DNA
gactcacatgaatcagtgcagcatcagatgtatgaccgaagcggacgaaggtgcgtgga

SEQ ID NO: 3 - DNA, 59 bp, synthetic construct
  source 1..59: mol_type = other DNA
gtggttcatcgcgtccgatatcaaacttcgtcaagtcgaagcggacgaaggtgcgtgga

SEQ ID NO: 4 - DNA, 59 bp, synthetic construct
  source 1..59: mol_type = other DNA
tactaggttgtacgatccctgcacttcagctaagcacgaagcggacgaaggtgcgtgga

SEQ ID NO: 5 - DNA, 59 bp, synthetic construct
  source 1..59: mol_type = other DNA
agctaccaatatttagtttccgagtctcagctcatgcgaagcggacgaaggtgcgtgga

SEQ ID NO: 6 - DNA, 36 bp, synthetic construct
  source 1..36: mol_type = other DNA
aaaaaaaaaaaagtccacgcaccttcgtccgcttcg

SEQ ID NO: 7-10 - skipped (4 entries)

SEQ ID NO: 11 - DNA, 58 bp, synthetic construct
  source 1..58: mol_type = other DNA
ccaacttgatattaataacactatagaccaccgcccgaagcggacgaaggtgcgtgga

SEQ ID NO: 12 - DNA, 58 bp, synthetic construct
  source 1..58: mol_type = other DNA
atgattaaactcctaagcagaaaacctaccgcgctcgaagcggacgaaggtgcgtgga

SEQ ID NO: 13 - DNA, 58 bp, synthetic construct
  source 1..58: mol_type = other DNA
tctttaataacctgattcagcgaaaccaatccgcgcgaagcggacgaaggtgcgtgga

SEQ ID NO: 14 - DNA, 58 bp, synthetic construct
  source 1..58: mol_type = other DNA
cggttatcgctggcgactccttcgagatggacgcccgaagcggacgaaggtgcgtgga

SEQ ID NO: 15 - DNA, 41 bp, synthetic construct
  source 1..41: mol_type = other DNA
aaaaaaaaaaaagtccacgcaccttcgtccgcttcgggcgg

SEQ ID NO: 16 - DNA, 41 bp, synthetic construct
  source 1..41: mol_type = other DNA
aaaaaaaaaaaagtccacgcaccttcgtccgcttcgagcgc

SEQ ID NO: 17 - DNA, 41 bp, synthetic construct
  source 1..41: mol_type = other DNA
aaaaaaaaaaaagtccacgcaccttcgtccgcttcgcgcgg

SEQ ID NO: 18 - DNA, 41 bp, synthetic construct
  source 1..41: mol_type = other DNA
aaaaaaaaaaaagtccacgcaccttcgtccgcttcgggcgt

SEQ ID NO: 19 - protein, 785 aa, synthetic construct
MILDADYITEDGKPIIRIFKKENGEFKVEYDRNFRPYIYALLRDDGAIDEIKKITAQRHGKVVRIVETEKIQRKFLGRPIEVWKLYLEHPQDQPAIRDKIREHQAVVDIFEYDIPFAKRYLIDKGLTPAEGNERLTFLAVAIAALYHEGEEFGKGPVIMISYADEEGAKVITWKKIDLPYVEVVSSEREMIKRLIRVIKEKDPDVIITYNGDNFDFPYLLKRAEKLGIKLLLGRDDNEPKIQKMGDSLAVEIKGRFHLDFLPVIRRTINLPTYTLEAVYKAFFGKPKDKVYADEIAKAWGTGEGLGRVAKYSMEDAKVTYELGREFFPMEAQLARLVGQPLWDVSRSSTGNLVEWFLLRKAYERNELAPNKPDEKEYERRLRESYEGGYVKEPEKGLWEGIVSLDFRSAGPSIIIAHNVSPDTLNREGCEEYDVAPKLGHRFCKDFPGFIPSLLGQLLEERQKIKKRMKESKDPVEKKPLDYRQRVIKILANSFYGYYGYAKARWYCKECAESVSAWGRQYIDLVRRELEARGFKVLYIDTDGLYATIPGVKDWEEVKRRALEFVDYINSKLPGVLELEYGGFYARGLFVTKKKYALIDEEGKIVTRGLEIVRRDWSEIAKETQARVLEAILKHGNVEELVKIVKDVTEKLTNYEVPPEKLVIYEQITRPINEYKAIGPHVAVAKRLMARGIKVKPGMVIGYIVLRGDGPISKRAISIEEFDPRKHKYDAEYYIENQVLPAVERILKAFGYKREDLRWQKTKQVGLGAWIKVKKSGGSGHHHHHH

SEQ ID NO: 20 - protein, 775 aa, synthetic construct
MILDADYITEDGKPIIRIFKKENGEFKVEYDRNFRPYIYALLRDDGAIDEIKKITAQRHGKVVRIVETEKIQRKFLGRPIEVWKLYLEHPQDQPAIRDKIREHQAVVDIFEYDIPFAKRYLIDKGLTPAEGNERLTFLAVAIAALYHEGEEFGKGPVIMISYADEEGAKVITWKKIDLPYVEVVSSEREMIKRLIRVIKEKDPDVIITYNGDNFDFPYLLKRAEKLGIKLLLGRDDNEPKIQKMGDSLAVEIKGRFHLDFLPVIRRTINLPTYTLEAVYKAFFGKPKDKVYADEIAKAWGTGEGLGRVAKYSMEDAKVTYELGREFFPMEAQLARLVGQPLWDVSRSSTGNLVEWFLLRKAYERNELAPNKPDEKEYERRLRESYEGGYVKEPEKGLWEGIVSLDFRSAGPSIIIAHNVSPDTLNREGCEEYDVAPKLGHRFCKDFPGFIPSLLGQLLEERQKIKKRMKESKDPVEKKPLDYRQRVIKILANSFYGYYGYAKARWYCKECAESVSAWGRQYIDLVRRELEARGFKVLYIDTDGLYATIPGVKDWEEVKRRALEFVDYINSKLPGVLELEYGGFYARGLFVTKKKYALIDEEGKIVTRGLEIVRRDWSEIAKETQARVLEAILKHGNVEELVKIVKDVTEKLTNYEVPPEKLVIYEQITRPINEYKAIGPHVAVAKRLMARGIKVKPGMVIGYIVLRGDGPISKRAISIEEFDPRKHKYDAEYYIENQVLPAVERILKAFGYKREDLRWQKTKQVGLGAWIKVKKS

SEQ ID NO: 21 - protein, 775 aa, synthetic construct
MILDADYITEDGKPIIRIFKKENGEFKVEYDRNFRPYIYALLRDDGAIDEIKKITAQRHGKVVRIVETEKIQRKFLGRPIEVWKLYLEHPQDQPAIRDKIREHQAVVDIFEYDIPFAKRYLIDKGLTPAEGNERLTFLAVAIAALYHEGEEFGKGPVIMISYADEEGAKVITWKKIDLPYVEVVSSEREMIKRLIRVIKEKDPDVIITYNGDNFDFPYLLKRAEKLGIKLLLGRDDNEPKIQKMGDSLAVEIKGRFHLDFLPVIRRTINLPTYTLEAVYKAFFGKPKDKVYADEIAKAWGTGEGLGRVAKYSMEDAKVTYELGREFFPMEAQLARLVGQPLWDVSRSSTGNLVEWFLLRKAYERNELAPNKPDEKEYERRLRESYEGGYVKEPEKGLWEGIVSLDFRSAGPSIIIAHNVSPDTLNREGSEEYDVAPKLGHRFSKDFPGFIPSLLGQLLEERQKIKKRMKESKDPVEKKPLDYRQRVIKILANSFYGYYGYAKARWYSKESAESVSAWGRQYIDLVRRELEARGFKVLYIDTDGLYATIPGVKDWEEVKRRALEFVDYINSKLPGVLELEYGGFYARGLFVTKKKYALIDEEGKIVTRGLEIVRRDWSEIAKETQARVLEAILKHGNVEELVKIVKDVTEKLTNYEVPPEKLVIYEQITRPINEYKAIGPHVAVAKRLMARGIKVKPGMVIGYIVLRGDGPISKRAISIEEFDPRKHKYDAEYYIENQVLPAVERILKAFGYKREDLRWQKTKQVGLGAWIKVKKS

SEQ ID NO: 22 - protein, 785 aa, synthetic construct
MILDADYITEDGKPIIRIFKKENGEFKVEYDRNFRPYIYALLRDDGAIDEIKKITAQRHGKVVRIVETEKIQRKFLGRPIEVWKLYLEHPQDQPAIRDKIREHQAVVDIFEYDIPFAKRYLIDKGLTPAEGNERLTFLAVAIAALYHEGEEFGKGPVIMISYADEEGAKVITWKKIDLPYVEVVSSEREMIKRLIRVIKEKDPDVIITYNGDNFDFPYLLKRAEKLGIKLLLGRDDNEPKIQKMGDSLAVEIKGRFHLDFLPVIRRTINLPTYTLEAVYKAFFGKPKDKVYADEIAKAWGTGEGLGRVAKYSMEDAKVTYELGREFFPMEAQLARLVGQPLWDVSRSSTGNLVEWFLLRKAYERNELAPNKPDEKEYERRLRESYEGGYVKEPEKGLWEGIVSLDFRSAGPSIIIAHNVSPDTLNREGSEEYDVAPKLGHRFSKDFPGFIPSLLGQLLEERQKIKKRMKESKDPVEKKPLDYRQRVIKILANSFYGYYGYAKARWYSKESAESVSAWGRQYIDLVRRELEARGFKVLYIDTDGLYATIPGVKDWEEVKRRALEFVDYINSKLPGVLELEYGGFYARGLFVTKKKYALIDEEGKIVTRGLEIVRRDWSEIAKETQARVLEAILKHGNVEELVKIVKDVTEKLTNYEVPPEKLVIYEQITRPINEYKAIGPHVAVAKRLMARGIKVKPGMVIGYIVLRGDGPISKRAISIEEFDPRKHKYDAEYYIENQVLPAVERILKAFGYKREDLRWQKTKQVGLGAWIKVKKSGGSGHHHHHH

(19) United States
(12) Patent Application Publication
(10) Pub. No.: US 2025/0354129 A1
(43) Pub. Date: Nov. 20, 2025

(54) HIGH FIDELITY SEQUENCING ENZYMES

(71) Applicant: Singular Genomics Systems, Inc., San Diego, CA (US)

(72) Inventors: Bruna Krebs Kutche, San Diego, CA (US); Joshua Catungal Corpuz, San Diego, CA (US); Eli N. Glezer, Del Mar, CA (US); Sumit Handa, San Diego, CA (US); Henry Hoi-Chung Kwan, San Diego, CA (US); Souad Naji, La Jolla, CA (US)

(21) Appl. No.: 19/208,361

(22) Filed: May 14, 2025

Related U.S. Application Data
(60) Provisional application No. 63/648,613, filed on May 16, 2024.

(51) Int. Cl. C12N 9/12; C12Q 1/6806

(52) U.S. Cl. CPC C12N 9/1252; C12Q 1/6806; C12Y 207/07007

(57) ABSTRACT

Disclosed herein, inter alia, are polymerases designed for accurate incorporation of nucleotides into a primer bound to a template polynucleotide.

### CROSS-REFERENCES TO RELATED APPLICATIONS

This application claims the benefit of U.S. Provisional Application No. 63/648,613, filed May 16, 2024, which is incorporated herein by reference in its entirety and for all purposes.

### SEQUENCE LISTING

The instant application contains a Sequence Listing which has been submitted electronically in XML format and is hereby incorporated by reference in its entirety. Said XML copy, created on May 7, 2025, is named 00623001US.xml, and is 24,576 bytes in size.

### BACKGROUND

Native DNA polymerases inherently exhibit a discriminatory behavior against modified nucleotides, which is evolutionarily advantageous as it ensures high fidelity during DNA replication. However, for sequencing applications that utilize reversible terminators, there is a need to adapt these enzymes to accept and efficiently incorporate such modified substrates. Achieving this requires a delicate balance through targeted mutations: the polymerase must be altered sufficiently to accommodate the structural peculiarities of reversible terminators without significantly compromising its intrinsic fidelity. Balancing incorporation kinetics and fidelity is a challenge. If the mutations in the polymerase result in a rapid average incorporation half-time but are too promiscuous such that the inappropriate nucleotide is incorporated into the primer, this will result in a large source of error in sequencing applications. Discovering a polymerase that has suitable kinetics and low misincorporation error remains a challenge. Disclosed herein, inter alia, are solutions to these and other problems in the art.

### BRIEF SUMMARY

In an aspect is provided a polymerase including an amino acid sequence that is at least 80% identical to a continuous 500 amino acid sequence within SEQ ID NO: 1. In embodiments, the polymerase includes a mutation at amino acid position 306 or an amino acid position corresponding to position 306. In embodiments, the mutation is aspartic acid, glutamine, asparagine, alanine, serine, proline, valine, or glycine.

### DETAILED DESCRIPTION

The aspects and embodiments described herein relate to high-fidelity sequencing enzymes and their use in a sequencing system.

### I. Definitions

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Unless defined otherwise, technical and scientific terms used herein have the same meaning as commonly understood by a person of ordinary skill in the art. Sec, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA with linear or circular framework. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

The term “duplex” in the context of polynucleotides refers, in the usual and customary sense, to double-strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like. Different polynucleotides may have different three-dimensional structures, and may perform various functions, known or unknown. Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The term “base” and “nucleobase” as used herein refers to a purine or pyrimidine compound, or a derivative thereof, that may be a constituent of nucleic acid (i.e. DNA or RNA, or a derivative thereof). In embodiments, the base is a derivative of a naturally occurring DNA or RNA base (e.g., a base analogue). In embodiments, the base is a base-pairing base. In embodiments, the base pairs to a complementary base. In embodiments, the base is capable of forming at least one hydrogen bond with a complementary base (e.g., adenine hydrogen bonds with thymine, adenine hydrogen bonds with uracil, guanine pairs with cytosine). Non-limiting examples of a base includes cytosine or a derivative thereof (e.g., cytosine analogue), guanine or a derivative thereof (e.g., guanine analogue), adenine or a derivative thereof (e.g., adenine analogue), thymine or a derivative thereof (e.g., thymine analogue), uracil or a derivative thereof (e.g., uracil analogue), hypoxanthine or a derivative thereof (e.g., hypoxanthine analogue), xanthine or a derivative thereof (e.g., xanthine analogue), guanosine or a derivative thereof (e.g., 7-methylguanosine analogue), deaza-adenine or a derivative thereof (e.g., deaza-adenine analogue), deaza-guanine or a derivative thereof (e.g., deaza-guanine), deaza-hypoxanthine or a derivative thereof, 5,6-dihydrouracil or a derivative thereof (e.g., 5,6-dihydrouracil analogue), 5-methylcytosine or a derivative thereof (e.g., 5-methylcytosine analogue), or 5-hydroxymethylcytosine or a derivative thereof (e.g., 5-hydroxymethylcytosine analogue) moieties. In embodiments, the base is thymine, cytosine, uracil, adenine, guanine, hypoxanthine, xanthine, theobromine, caffeine, uric acid, or isoguanine. In embodiments, the base is

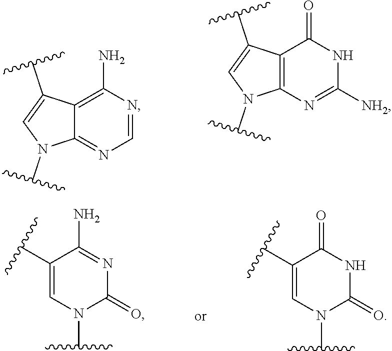 [SMILES: *c1cn(*)c2ncnc(N)c12 ; *c1cn(*)c2nc(N)[nH]c(=O)c12 ; *c1cn(*)c(=O)nc1N ; *c1cn(*)c(=O)[nH]c1=O]

A polynucleotide is typically composed of a specific sequence of four nucleotide bases: adenine (A); cytosine (C); guanine (G); and thymine (T) (uracil (U) for thymine (T) when the polynucleotide is RNA. Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “isolated”, when applied to a nucleic acid or protein, denotes that the nucleic acid or protein is essentially free of other cellular components with which it is associated in the natural state. It can be, for example, in a homogeneous state and may be in either a dry or an aqueous solution. Purity and homogeneity are typically determined using analytical chemistry techniques such as polyacrylamide gel electrophoresis or high performance liquid chromatography. A protein that is the predominant species present in a preparation is substantially purified.

The terms “analog” and “analogue” and “derivative” in reference to a chemical compound, refers to compounds having a structure similar to that of another one, but differing from it in respect of one or more different atoms, functional groups, or substructures that are replaced with one or more other atoms, functional groups, or substructures. In the context of a nucleotide useful in practicing the invention, a nucleotide analog refers to a compound that, like the nucleotide of which it is an analog, can be incorporated into a nucleic acid molecule (e.g., an extension product) by a suitable polymerase, for example, a DNA polymerase in the context of a dNTP analogue. The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

The term “complement,” as used herein, refers to a nucleotide (e.g., RNA or DNA) or a sequence of nucleotides capable of base pairing with a complementary nucleotide or sequence of nucleotides. As described herein and commonly known in the art, the complementary (matching) nucleoside of adenosine is thymidine and the complementary (matching) nucleoside of guanosine is cytidine. Thus, a complement may include a sequence of nucleotides that base pair with corresponding complementary nucleotides of a second nucleic acid sequence. The nucleotides of a complement may match, partially or completely, the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence, only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein, the complementarity of sequences may be partial, in which only some of the nucleic acids match according to base pairing, or complete, where all the nucleic acids match according to base pairing. Thus, two sequences that are complementary to each other may have a specified percentage of nucleotides that are complementary (i.e., about 60% identity, preferably 65%, 70%, 75%, 80%, 85%, 90%, 91%, 92%, 93%, 94%, 95%, 96%, 97%, 98%, 99%, or higher complementarity over a specified region).

“DNA” refers to deoxyribonucleic acid, a polymer of deoxyribonucleotides (e.g., dATP, dCTP, dGTP, dTTP, dUTP, etc.) linked by phosphodiester bonds. DNA can be single-stranded (ssDNA) or double-stranded (dsDNA), and can include both single and double-stranded (or “duplex”) regions. “RNA” refers to ribonucleic acid, a polymer of ribonucleotides linked by phosphodiester bonds. RNA can be single-stranded (ssRNA) or double-stranded (dsRNA), and can include both single and double-stranded (or “duplex”) regions. Single-stranded DNA (or regions thereof) and ssRNA can, if sufficiently complementary, hybridize to form double-stranded DNA/RNA complexes (or regions).

The term “primer” refers to any nucleic acid molecule that may hybridize to a template and be bound by a DNA polymerase and extended in a template-directed process for nucleic acid synthesis. The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment, the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

The term “DNA template” refers to any DNA molecule that may be bound by a DNA polymerase and utilized as a template for nucleic acid synthesis. In embodiments, the “DNA template” also refers to the DNA molecule that is subject to ligation by a ligase described herein.

The term “dATP analogue” refers to an analogue of deoxyadenosine triphosphate (dATP) that is a substrate for a DNA polymerase. The term “dCTP analogue” refers to an analogue of deoxycytidine triphosphate (dCTP) that is a substrate for a DNA polymerase. The term “dGTP analogue” refers to an analogue of deoxyguanosine triphosphate (dGTP) that is a substrate for a DNA polymerase. The term “dNTP analogue” refers to an analogue of deoxynucleoside triphosphate (dNTP) that is a substrate for a DNA polymerase. The term “dTTP analogue” refers to an analogue of deoxythymidine triphosphate (dUTP) that is a substrate for a DNA polymerase. The term “dUTP analogue” refers to an analogue of deoxyuridine triphosphate (dUTP) that is a substrate for a DNA polymerase.

The term “extendible” means, in the context of a nucleotide, primer, or extension product, that the 3′-OH group of the molecule is available and accessible to a DNA polymerase for extension or addition of nucleotides derived from dNTPs or dNTP analogues. “Incorporation” means joining of the modified nucleotide to the free 3′ hydroxyl group of a second nucleotide via formation of a phosphodiester linkage with the 5′ phosphate group of the modified nucleotide. The second nucleotide to which the modified nucleotide is joined will typically occur at the 3′ end of a polynucleotide chain.

The term “modified nucleotide” refers to nucleotide or nucleotide analogue modified in some manner. Typically, a nucleotide contains a single 5-carbon sugar moiety, a single nitrogenous base moiety and 1 to three phosphate moieties. In embodiments, a nucleotide can include a blocking moiety or a label moiety. A blocking moiety (e.g., a reversible terminator moiety) on a nucleotide prevents formation of a covalent bond between the 3′ hydroxyl moiety of the nucleotide and the 5′ phosphate of another nucleotide. A blocking moiety on a nucleotide can be reversible (i.e., a reversible terminator), whereby the blocking moiety can be removed or modified to allow the 3′ hydroxyl to form a covalent bond with the 5′ phosphate of another nucleotide. A blocking moiety can be effectively irreversible under particular conditions used in a method set forth herein. A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both.

A “removable” group, e.g., a label or a blocking group or protecting group, refers to a chemical group that can be removed from a dNTP analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a dNTP of dNTP analogue.

“Reversible blocking groups” or “reversible terminators” include a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. Suitable nucleotide blocking moieties are described in applications WO 2004/018497, U.S. Pat. Nos. 7,057,026, 7,541,444, WO 96/07669, U.S. Pat. Nos. 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group-OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethy reversible terminator.

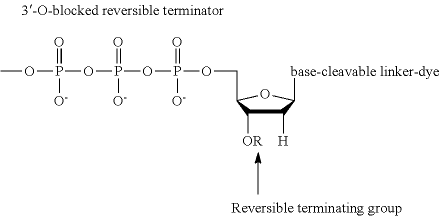

The term “non-covalent linker” is used in accordance with its ordinary meaning and refers to a divalent moiety which includes at least two molecules that are not covalently linked to each other but are capable of interacting with each other via a non-covalent bond (e.g. electrostatic interactions (e.g. ionic bond, hydrogen bond, halogen bond) or van der Waals interactions (e.g. dipole-dipole, dipole-induced dipole, London dispersion). In embodiments, the non-covalent linker is the result of two molecules that are not covalently linked to each other that interact with each other via a non-covalent bond.

The terms “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), hydrazine (N₂H₄)). A chemically cleavable linker is non-enzymatically cleavable. In embodiments, the cleavable linker is cleaved by contacting the cleavable linker with a cleaving agent. In embodiments, the cleaving agent is sodium dithionite (Na₂S₂O₄), weak acid, hydrazine (N₂H₄), Pd(0), or light-irradiation (e.g., ultraviolet radiation).

The term “orthogonal detectable label” or “orthogonal detectable moiety” as used herein refer to a detectable label (e.g. fluorescent dye or detectable dye) that is capable of being detected and identified (e.g., by use of a detection means (e.g., emission wavelength, physical characteristic measurement)) in a mixture or a panel (collection of separate samples) of two or more different detectable labels. For example, two different detectable labels that are fluorescent dyes are both orthogonal detectable labels when a panel of the two different fluorescent dyes is subjected to a wavelength of light that is absorbed by one fluorescent dye but not the other and results in emission of light from the fluorescent dye that absorbed the light but not the other fluorescent dye. Orthogonal detectable labels may be separately identified by different absorbance or emission intensities of the orthogonal detectable labels compared to each other and not only be the absolute presence of absence of a signal. An example of a set of four orthogonal detectable labels is the set of Rox™-Labeled Tetrazine, Alexa Fluor® 488-Labeled SHA, Cy®5-Labeled Streptavidin, and R6G-Labeled Dibenzocyclooctyne. ROX™ is a trademark of Applera Corporation. Alexa Fluor® is a trademark of Life Technologies Corporation. Cy® is a trademark of Cytiva.

A “detectable agent” or “detectable compound” or “detectable label” or “detectable moiety” is a composition detectable by spectroscopic, photochemical, biochemical, immunochemical, chemical, magnetic resonance imaging, or other physical means. For example, detectable agents include fluorophores (e.g. fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. Examples of detectable agents include imaging agents, including fluorescent and luminescent substances, including, but not limited to, a variety of organic or inorganic small molecules commonly referred to as “dyes,” “labels,” or “indicators.” Examples include fluorescein, rhodamine, acridine dyes, Alexa Fluor® dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescein isothiocyanate moiety, tetramethylrhodamine-5- (and 6)-isothiocyanate moiety, Cy®2 moiety, Cy®3 moiety, Cy®5 moiety, Cy®7 moiety, 4′,6-diamidino-2-phenylindole moiety, Hoechst 33258 moiety, Hoechst 33342 moiety, Hoechst 34580 moiety, propidium-iodide moiety, or acridine orange moiety. In embodiments, the detectable label is a fluorescent dye. In embodiments, the detectable label is a fluorescent dye capable of exchanging energy with another fluorescent dye (e.g., fluorescence resonance energy transfer (FRET) chromophores).

A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics, which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide, also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following groups each contain amino acids that are conservative substitutions for one another: 1) Non-polar-Alanine (A), Leucine (L), Isoleucine (I), Valine (V), Glycine (G), Methionine (M); 2) Aliphatic-Alanine (A), Leucine (L), Isoleucine (I), Valine (V); 3) Acidic-Aspartic acid (D), Glutamic acid (E); 4) Polar-Asparagine (N), Glutamine (Q); Serine(S), Threonine (T); 5) Basic-Arginine (R), Lysine (K); 7) Aromatic-Phenylalanine (F), Tyrosine (Y), Tryptophan (W), Histidine (H); 8) Other-Cysteine (C) and Proline (P).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Percent identity often refers to the percentage of matching positions of two sequences for a contiguous section of positions, wherein the two sequences are aligned in such a way to maximize matching positions and minimize gaps of non-matching positions. In some embodiments, alignments are conducted wherein there are no gaps between the two sequences. In some instances, the alignment results in less than 5% gaps, less than 3% gaps, or less than 1% gaps. Additional methods of sequence comparison or alignment are also consistent with the disclosure.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST® or BLAST® 2.0 sequence comparison algorithm with default parameters described below, or by manual alignment and visual inspection (see, e.g., NCBI web site www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length. As used herein, percent (%) amino acid sequence identity is defined as the percentage of amino acids in a candidate sequence that is identical to the amino acids in a reference sequence, after aligning the sequences and introducing gaps, if necessary, to achieve the maximum percent sequence identity. Alignment for purposes of determining percent sequence identity can be achieved in various ways that are within the level of skill in the art, for instance, using publicly available computer software such as BLAST®, BLAST®-2, ALIGN, ALIGN-2 or Megalign (DNASTAR) software. Appropriate parameters for measuring alignment, including any algorithms needed to achieve maximal alignment over the full length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 10 to 700, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “position”, “numbered with reference to” or “corresponding to,” when used in the context of the numbering of a given amino acid or polynucleotide sequence, refer to the numbering of the residues of a specified reference sequence when the given amino acid or polynucleotide sequence is compared to the reference sequence. Similarly, the term “functionally equivalent to” in relation to an amino acid position refers to an amino acid residue in a protein that corresponds to a particular amino acid in a reference sequence. An amino acid “corresponds” to a given residue when it occupies the same essential structural position within the protein as the given residue. One skilled in the art will immediately recognize the identity and location of residues corresponding to a specific position in a protein (e.g., ligase) in other proteins with different numbering systems. For example, by performing a simple sequence alignment with a protein (e.g., ligase) the identity and location of residues corresponding to specific positions of the protein are identified in other protein sequences aligning to the protein. For example, a selected residue in a selected protein corresponds to cysteine at position 22 when the selected residue occupies the same essential spatial or other structural relationship as a cysteine at position 22. In some embodiments, where a selected protein is aligned for maximum homology with a protein, the position in the aligned selected protein aligning with cysteine 22 is said to correspond to cysteine 22. Instead of a primary sequence alignment, a three-dimensional structural alignment can also be used, e.g., where the structure of the selected protein is aligned for maximum correspondence with the cysteine at position 22, and the overall structures compared. In this case, an amino acid that occupies the same essential position as cysteine 22 in the structural model is said to correspond to the cysteine 22 residue. Sequence alignments may be compiled using any of the standard alignment tools known in the art, such as for example BLAST® and DIAMOND (Buchfink et al. Nat Methods 12, 59-60 (2015)), and the like.

The term “DNA polymerase” and “nucleic acid polymerase” are used in accordance with their plain ordinary meaning and refer to enzymes capable of synthesizing nucleic acid molecules from nucleotides (e.g., deoxyribonucleotides). Typically, a DNA polymerase adds nucleotides to the 3′ end of a DNA strand one nucleotide at a time.

The term “exonuclease activity” is used in accordance with its ordinary meaning in the art, and refers to the removal of a nucleotide from a nucleic acid by a DNA polymerase. For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide, thereby releasing deoxyribonucleoside 5′-monophosphates one after another. One having skill in the art understands that an enzyme having 3′-5′ exonuclease activity does not cleave DNA strands without terminal 3′-OH moieties. In embodiments, 3′-5′ exonuclease activity refers to the successive removal of nucleotides in single-stranded DNA in a 3′->5′ direction, releasing deoxyribonucleoside 5′-monophosphates one after another. Methods for quantifying exonuclease activity are known in the art, see for example Southworth et al, PNAS Vol 93, 8281-8285 (1996).

The terms “measure”, “measuring”, “measurement” and the like refer not only to quantitative measurement of a particular variable, but also to qualitative and semi-quantitative measurements. Accordingly, “measurement” also includes detection, meaning that merely detecting a change, without quantification, constitutes measurement.

A “polymerase-template complex” refers to a functional complex between a DNA polymerase and a DNA primer-template molecule (e.g., nucleic acid). In embodiments, the polymerase is non-covalently bound to a nucleic acid primer and the template nucleic acid molecule.

The terms “sequencing”, “sequence determination”, “determining a nucleotide sequence”, and the like include determination of partial as well as full sequence information of the polynucleotide being sequenced. That is, the term includes sequence comparisons, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide.

The term “sequencing reaction mixture” refers to an aqueous mixture that contains the reagents necessary to allow a dNTP or dNTP analogue to add a nucleotide to a DNA strand by a DNA polymerase. Exemplary mixtures include buffers (e.g., saline-sodium citrate (SSC), tris(hydroxymethyl)aminomethane or “Tris”), salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), detergents and/or crowding agents or stabilizers (e.g., PEG, Tween®, BSA). Tween® is a registered trademark of Croda International PLC.

As used herein, the terms “solid support” and “substrate” and “substrate surface” and “solid surface” refers to discrete surfaces that are solid or semi-solid. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may include a discrete particle that may be spherical (e.g., microspheres) or have a non-spherical or irregular shape, such as cubic, cuboid, pyramidal, cylindrical, conical, oblong, or disc-shaped, and the like. A bead can be non-spherical in shape. A solid support may be used interchangeably with the term “bead.” A solid support may further include a polymer or hydrogel on the surface to which the primers are attached. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zconor®, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located on a microplate. Particularly useful solid supports for some embodiments have at least one surface located on a microplate within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of a substantially circular particle to maximize the contact between the particle. In embodiments, the wells of an array are randomly located such that nearest neighbor wells have random spacing between each other. Alternatively, in embodiments the spacing between the wells can be ordered, for example, forming a regular pattern. The term solid substrate is encompassing of a substrate (e.g., a microplate or flow cell) having a surface including a polymer coating covalently attached thereto.

Broadly speaking, for nucleic acid sequencing applications, a flow cell may be considered a reaction chamber that contains one or more nucleic acid templates tethered to a solid support, to which nucleotides and ancillary reagents are iteratively applied and washed away. The flow cell allows for imaging of the sites at which the nucleic acids are bound, and resulting image data is used for the desired analysis. The latest commercial sequencing instruments use flow cells and massive parallelization to increase sequencing capacity.

In embodiments, the solid substrate is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS®, hematite, AMPure XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, a flow cell includes inlet and outlet ports and a flow channel extending there between.

As used herein, the term “channel” refers to a passage in or on a substrate material that directs the flow of a fluid. A channel may run along the surface of a substrate, or may run through the substrate between openings in the substrate. A channel can have a cross section that is partially or fully surrounded by substrate material (e.g., a fluid impermeable substrate material). For example, a partially surrounded cross section can be a groove, trough, furrow or gutter that inhibits lateral flow of a fluid. The transverse cross section of an open channel can be, for example, U-shaped, V-shaped, curved, angular, polygonal, or hyperbolic. A channel can have a fully surrounded cross section such as a tunnel, tube, or pipe. A fully surrounded channel can have a rounded, circular, elliptical, square, rectangular, or polygonal cross section. In particular embodiments, a channel can be located in a flow cell, for example, being embedded within the flow cell. A channel in a flow cell can include one or more windows that are transparent to light in a particular region of the wavelength spectrum. In embodiments, the channel is filled by the one or more polymers, and flow through the channel (e.g., as in a sample fluid) is directed through the polymer in the channel. In embodiments, the tissue is in a channel of a flow cell.

The term “array” as used herein, refers to a container (e.g., a multiwell container, reaction vessel, or flow cell) including a plurality of features (e.g., wells). For example, an array may include a container with a plurality of wells. In embodiments, the array is a microplate. In embodiments, the array is a flow cell.

The term “microplate,” “microtiter plate,” or “multiwell plate” as used herein, refers to a substrate including a surface, the surface including a plurality of chambers or wells separated from each other by interstitial regions on the surface. In embodiments, the microplate has dimensions as provided and described by American National Standards Institute (ANSI) and Society for Laboratory Automation And Screening (SLAS); for example the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference. The dimensions of the microplate as described herein and the arrangement of the reaction chambers may be compatible with an established format for automated laboratory equipment. In embodiments, the device described herein provides methods for high-throughput screening. High-throughput screening (HTS) refers to a process that uses a combination of modern robotics, data processing and control software, liquid handling devices, and/or sensitive detectors, to efficiently process a large amount of (e.g., thousands, hundreds of thousands, or millions) samples in biochemical, genetic, or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins, or polynucleotides in a cell.

The reaction chambers may be provided as wells, for example an array or microplate may contain 2, 4, 6, 12, 24, 48, 96, 384, or 1536 sample wells. In embodiments, the 96 and 384 wells are arranged in a 2:3 rectangular matrix. In embodiments, the 24 wells are arranged in a 3:8 rectangular matrix. In embodiments, the 48 wells are arranged in a 3:4 rectangular matrix. In embodiments, the reaction chamber is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). In embodiments, the microplate is about 5 inches by about 3.33 inches, and includes a plurality of 5 mm diameter wells. In embodiments, the microplate is about 5 inches by about 3.33 inches, and includes a plurality of 6 mm diameter wells. In embodiments, the microplate is about 5 inches by about 3.33 inches, and includes a plurality of 7 mm diameter wells. In embodiments, the microplate is about 5 inches by about 3.33 inches, and includes a plurality of 7.5 mm diameter wells. In embodiments, the microplate is 5 inches by 3.33 inches, and includes a plurality of 7.5 mm diameter wells. In embodiments, the microplate is about 5 inches by about 3.33 inches, and includes a plurality of 8 mm diameter wells. In embodiments, the microplate is a flat glass or plastic tray in which an array of wells are formed, wherein each well can hold between from a few microliters to hundreds of microliters of fluid reagents and samples.

The term “species”, when used in the context of describing a particular compound or molecule species, refers to a population of chemically indistinct molecules. When used in the context of taxonomy, “species” is the basic unit of classification and a taxonomic rank.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

“Control” or “control experiment” is used in accordance with its plain ordinary meaning and refers to an experiment in which the subjects (e.g., enzymes) or reagents of the experiment are treated as in a parallel experiment except for omission of a procedure, reagent, or variable of the experiment (e.g., a ligase not having one or more mutations relative to the polymerase being tested). In some instances, the control is used as a standard of comparison in evaluating experimental effects. In some embodiments, a control is the measurement of the activity of a protein in the absence of a mutation as described herein (including embodiments and examples). “Control ligase” is defined herein as the ligase against which the activity of the altered ligase is compared. Unless otherwise stated, by “wild type” it is generally meant that the ligase comprises its natural amino acid sequence, as it would be found in nature. The invention is not limited to merely a comparison of activity of the ligase as described herein against the wild type. Many ligases exist whose amino acid sequence has been modified (e.g., by amino acid substitution mutations) and which can prove to be a suitable control for use in assessing the ligation efficiencies of the ligases as described herein. The control ligase can, therefore, include any known ligase, including mutant ligases known in the art. The activity of the chosen “control” ligase with respect to the ligation of single-stranded DNA polynucleotides may be determined by a ligation activity assay as described infra. In embodiments, the control includes performing the experiment with a wild type ligase.

The term “modulate” is used in accordance with its plain ordinary meaning and refers to the act of changing or varying one or more properties. “Modulation” refers to the process of changing or varying one or more properties.

The term “kit” is used in accordance with its plain ordinary meaning and refers to any delivery system for delivering materials or reagents for carrying out a method of the invention. Such delivery systems include systems that allow for the storage, transport, or delivery of reaction reagents (e.g., nucleotides, enzymes, nucleic acid templates, etc. in the appropriate containers) and/or supporting materials (e.g., buffers, written instructions for performing the reaction, etc.) from one location to another location. For example, kits include one or more enclosures (e.g., boxes) containing the relevant reaction reagents and/or supporting materials. Such contents may be delivered to the intended recipient together or separately. For example, a first container may contain an enzyme, while a second container contains nucleotides. In embodiments, the kit includes vessels containing one or more enzymes, primers, adaptors, or other reagents as described herein. Vessels may include any structure capable of supporting or containing a liquid or solid material and may include tubes, vials, jars, containers, tips, etc. In embodiments, a wall of a vessel may permit the transmission of light through the wall. In embodiments, the vessel may be optically clear. The kit may include the enzyme and/or nucleotides in a buffer. In embodiments, the buffer includes an acetate buffer, 3-(N-morpholino) propanesulfonic acid (MOPS) buffer, N-(2-Acetamido)-2-aminocthanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminocthanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

The phrase “stringent hybridization conditions” refers to conditions under which a primer will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 65° C.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

As used herein, the terms “biomolecule” or “analyte” refer to an agent (e.g., a compound, macromolecule, or small molecule), and the like derived from a biological system (e.g., an organism, a cell, or a tissue). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, proteins, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). The biomolecule may be any substance (e.g. molecule) or entity that is desired to be detected by the method of the invention. The biomolecule is the “target” of the assay method of the invention. The biomolecule may accordingly be any compound that may be desired to be detected, for example a peptide or protein, or nucleic acid molecule or a small molecule, including organic and inorganic molecules. The biomolecule may be a cell or a microorganism, including a virus, or a fragment or product thereof. Biomolecules of particular interest may thus include proteinaceous molecules such as peptides, polypeptides, proteins or prions or any molecule which includes a protein or polypeptide component, etc., or fragments thereof. The biomolecule may be a single molecule or a complex that contains two or more molecular subunits, which may or may not be covalently bound to one another, and which may be the same or different. Thus, in addition to cells or microorganisms, such a complex biomolecule may also be a protein complex. Such a complex may thus be a homo- or hetero-multimer. Aggregates of molecules e.g., proteins may also be target analytes, for example aggregates of the same protein or different proteins. The biomolecule may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules.

As used herein, “biomaterial” refers to any biological material produced by an organism. In some embodiments, biomaterial includes secretions, extracellular matrix, proteins, lipids, organelles, membranes, cells, portions thereof, and combinations thereof. In some embodiments, cellular material includes secretions, extracellular matrix, proteins, lipids, organelles, membranes, cells, portions thereof, and combinations thereof. In some embodiments, biomaterial includes viruses. In some embodiments, the biomaterial is a replicating virus and thus includes virus infected cells. In embodiments, a biological sample includes biomaterials.

As used herein, the term “primed template DNA molecule” refers to a template DNA molecule which is associated with a primer (a short polynucleotide) that can serve as a starting point for DNA synthesis.

As used herein, the term “incorporating a nucleotide into a nucleic acid sequence” refers to the process of joining a cognate nucleotide to a nucleic acid primer by formation of a phosphodiester bond. In embodiments, methods of incorporating a nucleotide into a nucleic acid sequence includes combining in a reaction vessel: (i) a nucleic acid template, (ii) a nucleotide solution comprising a plurality of nucleotides, and (iii) a polymerase.

As used herein, the term “primer-template hybridization complex” refers to a double stranded nucleic acid complex formed as a result of a hybridization event between a DNA template molecule and a primer. In embodiments, the formation of a template complex enables elongation at the 3′ end of the primer.

A nucleic acid can be amplified by a suitable method. The term “amplified” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer). Amplification according to the present disclosure encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction—CCR), and the like.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and optionally denaturing the newly-formed nucleic acid duplex to separate the strands. The cycle may or may not be repeated. Amplification can include thermocycling or can be performed isothermally.

As used herein, the term “rolling circle amplification (RCA)” refers to a nucleic acid amplification reaction that amplifies a circular nucleic acid template (e.g., single-stranded DNA circles) via a rolling circle process. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in vitro under isothermal conditions using a suitable nucleic acid polymerase.

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm 2, at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Provided herein are methods and compositions for analyzing a sample (e.g., sequencing nucleic acids within a sample). A sample (e.g., a sample including nucleic acid) can be obtained from a suitable subject. In embodiments, the polymerase may be introduced into the sample, in situ. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, car, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, car, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid).

In some embodiments, a sample includes one or more nucleic acids, or fragments thereof. A sample can include nucleic acids obtained from one or more subjects. In some embodiments, the nucleic acid is in a cell or tissue. In some embodiments, the nucleic acid is obtained from a cell or tissue. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

As used herein, the term “polynucleotide-binding polypeptide” refers to an independently folded protein domain that includes a structural motif that is capable of recognizing and binding to a double-stranded or single-stranded polynucleotide. A polynucleotide-binding polypeptide is capable of recognizing specific polynucleotide sequences, which enables the polynucleotide-binding polypeptide to bind to the double-stranded polynucleotide or single-stranded polynucleotide with high affinity and specificity. Structural examples of a polynucleotide-binding polypeptide includes but is not limited to, helix-turn-helix domain, zinc finger domain, leucine zipper domain, and helix-loop-helix domain. Described herein are methods and compositions directed to a recombinant ligase attached to a polynucleotide-binding polypeptide. Exemplary examples of polynucleotide-binding polypeptide include, but are not limited to, Ss07d, hLig3 zinc finger, Sac7d, and Sac7e (see, e.g., Kalichuk et al. Sci Rep. 2016 Nov. 17:6:37274 and Bauer et al. PLOS One. 2017 Dec. 28; 12(12):e0190062, each of which are incorporated herein by reference in their entirety).

“Histidine-tag” or “His-tag” refers to a polypeptide sequence comprising between two (His₂) and ten (His₁₀) consecutive histidine residues. In embodiments, the His-tag facilitates affinity purification of recombinant proteins by enabling specific binding to metal ions, such as nickel (Ni²⁺) or cobalt (Co²⁺), immobilized on chromatographic resins (e.g., immobilized metal affinity chromatography, IMAC). In embodiments, the His-tag may be positioned at the N-terminus, the C-terminus, or within an internal region of a target protein, depending on the design of the expression construct. In embodiments, the His-tag facilitates purification, detection, or immobilization of the tagged protein while minimally affecting its biological function.

### II. Compositions & Kits

Provided herein are compositions including mutant polypeptides (i.e., mutant polymerases) exhibiting increased incorporation of nucleotides relative to a control (e.g., wildtype polymerase). Mutations in the polymerases described herein variously include one or more changes to amino acid residues present in the polypeptide sequence. Additions, substitutions, or deletions are all examples of mutations that are used to generate mutant polypeptides. Substitutions in some instances include the exchange of one amino acid for an alternative amino acid, and such alternative amino acids differ from the original amino acid with regard to size, shape, conformation, or chemical structure. Mutations in some instances are conservative or non-conservative. Conservative mutations comprise the substitution of an amino acid with an amino acid that possesses similar chemical properties. Additions often comprise the insertion of one or more amino acids at the N-terminal, C-terminal, or internal positions of the polypeptide. In some embodiments, additions include fusion polypeptides, wherein one or more additional polypeptides (i.e., a polypeptide from a different source) is connected (e.g., covalently linked to the N- or C-terminus) to the polymerase as described herein. Such additional polypeptides include domains with additional activity, or sequences with additional function (e.g., improve expression, aid purification, improve solubility, attach to a solid support, or other function).

Provided herein are, inter alia, modified Pyrococcus Family B DNA polymerases. Family B polymerases characteristically have separate domains for DNA polymerase activity and 3′-5′ exonuclease activity. The exonuclease domain is characterized by as many as six and at least three conserved amino acid sequence motifs in and around a structural binding pocket. During polymerization, nucleotides are added to the 3′ end of the primer strand and during the 3′-5′ exonuclease reaction, the 3′ terminus of the primer is shifted to the 3′-5′ exonuclease domain and the one or more of the 3′-terminal nucleotides are hydrolyzed. In embodiments, the variants of a Pyrococcus family B DNA polymerase provided herein have detectable strand displacing activity and are useful in methods of incorporating modified nucleotides in nucleic acid synthesis reactions. In embodiments, the polymerase is a thermophilic nucleic acid polymerase.

Parent archacal polymerases may be DNA polymerases that are isolated from naturally occurring organisms. The parent DNA polymerases, also referred to as wild type polymerase, share the property of having a structural binding pocket that binds and hydrolyzes a substrate nucleic acid, producing 5′-dNMP. The structural binding pocket in this family of polymerases also shares the property of having sequence motifs that form the binding pocket, referred to as Exo Motifs I-VI. In embodiments, the parent or wild type P. horikoshii polymerase has an amino acid sequence comprising SEQ ID NO: 1. In embodiments, the polymerase has one or more amino acid substitution mutations relative to SEQ ID NO: 1.

In embodiments, the polymerase (a synthetic or variant DNA polymerase) provided herein may contain 1, 2, 3, 4, 5, 6, 7, 8, 9, 10, 11, 12, 13, 14, 15, or more mutations as compared to the wild-type sequence of SEQ ID NO: 1. The polymerase (a synthetic or variant DNA polymerase) may contain 10, 20, 30, 40, 50 or more mutations as compared to the wild-type sequence of SEQ ID NO: 1. The polymerase (a synthetic or variant DNA polymerase) may contain between 10 and 20 (inclusive of endpoints, e.g., 10, 41 . . . 49, and 20), between 20 and 30, between 30 and 40, or between 40 or 50 mutations as compared to SEQ ID NO: 1.

In embodiments, the polymerase includes an amino acid sequence that is at least 85% identical to a continuous 500 amino acid sequence within SEQ ID NO: 1. In embodiments, the polymerase includes an amino acid sequence that is at least 90% identical to a continuous 500 amino acid sequence within SEQ ID NO: 1. In embodiments, the polymerase includes an amino acid sequence that is at least 95% identical to a continuous 500 amino acid sequence within SEQ ID NO: 1. In embodiments, the polymerase includes an amino acid sequence that is at least 98% identical to a continuous 500 amino acid sequence within SEQ ID NO: 1. In embodiments, the polymerase includes an amino acid sequence that is at least 99% identical to a continuous 500 amino acid sequence within SEQ ID NO: 1. In embodiments, the polymerase includes an amino acid sequence that is 90% identical to a continuous 500 amino acid sequence within SEQ ID NO: 1. In embodiments, the polymerase includes an amino acid sequence that is 95% identical to a continuous 500 amino acid sequence within SEQ ID NO: 1. In embodiments, the polymerase includes an amino acid sequence that is 90% identical to SEQ ID NO: 1. In embodiments, the polymerase includes an amino acid sequence that is 95% identical to SEQ ID NO: 1.

In an aspect is a polymerase including an amino acid sequence that is at least 50%, 60%, 70%, 80%, 85%, 90%, 95%, or at least 99% identical to a continuous 500 amino acid sequence within SEQ ID NO: 1; including a mutation at amino acid position 306 or an amino acid position corresponding to position 306. In embodiments, the mutation is aspartic acid, glutamine, asparagine, alanine, serine, proline, valine, or glycine. In embodiments, the mutation is aspartic acid. In embodiments, the mutation is glutamine. In embodiments, the mutation is asparagine. In embodiments, the mutation is alanine. In embodiments, the mutation is serine. In embodiments, the mutation is proline. In embodiments, the mutation is valine. In embodiments, the mutation is glycine.

In embodiments, mutations may include substitution of the amino acid in the parent amino acid sequences with an amino acid, which is not the parent amino acid. In embodiments, the mutations may result in conservative amino acid changes. In embodiments, non-polar amino acids may be converted into polar amino acids (threonine, asparagine, glutamine, cysteine, tyrosine, aspartic acid, glutamic acid or histidine) or the parent amino acid may be changed to an alanine. Wild type polymerase sequences are typical initial sequences for protein or enzyme engineering to generate mutant polymerases. In some embodiments, a polypeptide differs from a wild-type sequence (naturally occurring) by at least one amino acid. Any number of mutations is introduced into a polypeptide or portion of a polypeptide described herein, such as 1, 2, 3, 4, 5, 6, 7, 8, 9, 10, 20, 50, or more than 50 mutations. In embodiments, the polymerase differs from a wild-type sequence by at least two amino acids. In embodiments, the polymerase differs from a wild-type sequence by at least three, four, five, or at least six amino acids.

In embodiments, the mutation at amino acid position 306 or an amino acid position corresponding to position 306 is glycine, alanine, or valine. In embodiments, the mutation at amino acid position 306 is glycine. In embodiments, the mutation at amino acid position 306 or an amino acid position corresponding to position 306 is glycine. In embodiments, the mutation at amino acid position 306 or an amino acid position corresponding to position 306 is alanine. In embodiments, the mutation at amino acid position 306 or an amino acid position corresponding to position 306 is valine. In embodiments, the mutation at amino acid position 306 is glycine. In embodiments, the mutation at amino acid position 306 is alanine. In embodiments, the mutation at amino acid position 306 is valine.

In embodiments, the polymerase includes a leucine, isoleucine, valine, alanine, or glycine at amino acid position 341 or an amino acid position corresponding to position 341. In embodiments, the polymerase includes a leucine at amino acid position 341 or an amino acid position corresponding to position 341. In embodiments, the polymerase includes an isoleucine at amino acid position 341 or an amino acid position corresponding to position 341. In embodiments, the polymerase includes a valine at amino acid position 341 or an amino acid position corresponding to position 341. In embodiments, the polymerase includes an alanine at amino acid position 341 or an amino acid position corresponding to position 341. In embodiments, the polymerase includes a glycine at amino acid position 341 or an amino acid position corresponding to position 341. In embodiments, the polymerase includes a leucine at amino acid position 341. In embodiments, the polymerase includes an isoleucine at amino acid position 341. In embodiments, the polymerase includes a valine at amino acid position 341. In embodiments, the polymerase includes an alanine at amino acid position 341. In embodiments, the polymerase includes a glycine at amino acid position 341.

In embodiments, the polymerase includes a tyrosine, phenylalanine, tryptophan, leucine, isoleucine, or valine at amino acid position 494 or an amino acid position corresponding to position 494. In embodiments, the polymerase includes a tyrosine at amino acid position 494 or an amino acid position corresponding to position 494. In embodiments, the polymerase includes a phenylalanine at amino acid position 494 or an amino acid position corresponding to position 494. In embodiments, the polymerase includes a tryptophan at amino acid position 494 or an amino acid position corresponding to position 494. In embodiments, the polymerase includes a leucine at amino acid position 494 or an amino acid position corresponding to position 494. In embodiments, the polymerase includes an isoleucine at amino acid position 494 or an amino acid position corresponding to position 494. In embodiments, the polymerase includes a valine at amino acid position 494 or an amino acid position corresponding to position 494. In embodiments, the polymerase includes a tyrosine at amino acid position 494. In embodiments, the polymerase includes a phenylalanine at amino acid position 494. In embodiments, the polymerase includes a tryptophan at amino acid position 494. In embodiments, the polymerase includes a leucine at amino acid position 494. In embodiments, the polymerase includes an isoleucine at amino acid position 494. In embodiments, the polymerase includes a valine at amino acid position 494.

In embodiments, the polymerase includes glutamic acid, aspartic acid, glutamine, asparagine, alanine, serine, proline, valine, or glycine at amino acid position 581 or an amino acid position corresponding to position 581. In embodiments, the polymerase includes glutamic acid at amino acid position 581 or an amino acid position corresponding to position 581. In embodiments, the polymerase includes aspartic acid at amino acid position 581 or an amino acid position corresponding to position 581. In embodiments, the polymerase includes glutamine at amino acid position 581 or an amino acid position corresponding to position 581. In embodiments, the polymerase includes asparagine at amino acid position 581 or an amino acid position corresponding to position 581. In embodiments, the polymerase includes alanine at amino acid position 581 or an amino acid position corresponding to position 581. In embodiments, the polymerase includes serine at amino acid position 581 or an amino acid position corresponding to position 581. In embodiments, the polymerase includes proline at amino acid position 581 or an amino acid position corresponding to position 581. In embodiments, the polymerase includes valine at amino acid position 581 or an amino acid position corresponding to position 581. In embodiments, the polymerase includes glycine at amino acid position 581 or an amino acid position corresponding to position 581.

In embodiments, the polymerase includes glutamic acid at amino acid position 581. In embodiments, the polymerase includes aspartic acid at amino acid position 581. In embodiments, the polymerase includes glutamine at amino acid position 581. In embodiments, the polymerase includes asparagine at amino acid position 581. In embodiments, the polymerase includes alanine at amino acid position 581. In embodiments, the polymerase includes serine at amino acid position 581. In embodiments, the polymerase includes proline at amino acid position 581. In embodiments, the polymerase includes valine at amino acid position 581. In embodiments, the polymerase includes glycine at amino acid position 581.

In embodiments, the polymerase includes a tyrosine, phenylalanine, tryptophan, leucine, isoleucine, or valine at amino acid position 588 or an amino acid position corresponding to position 588. In embodiments, the polymerase includes a tyrosine at amino acid position 588 or an amino acid position corresponding to position 588. In embodiments, the polymerase includes a phenylalanine at amino acid position 588 or an amino acid position corresponding to position 588. In embodiments, the polymerase includes a tryptophan at amino acid position 588 or an amino acid position corresponding to position 588. In embodiments, the polymerase includes a leucine at amino acid position 588 or an amino acid position corresponding to position 588. In embodiments, the polymerase includes an isoleucine at amino acid position 588 or an amino acid position corresponding to position 588. In embodiments, the polymerase includes a valine at amino acid position 588 or an amino acid position corresponding to position 588. In embodiments, the polymerase includes tyrosine at amino acid position 588. In embodiments, the polymerase includes phenylalanine at amino acid position 588. In embodiments, the polymerase includes a tryptophan at amino acid position 588. In embodiments, the polymerase includes a leucine at amino acid position 588. In embodiments, the polymerase includes isoleucine at amino acid position 588. In embodiments, the polymerase includes valine at amino acid position 588.

In embodiments, the polymerase includes lysine, arginine, histidine, glutamic acid, aspartic acid, glutamine, asparagine, alanine, serine, proline, valine, or glycine at amino acid position 280 or an amino acid position corresponding to position 280. In embodiments, the polymerase includes lysine at amino acid position 280 or an amino acid position corresponding to position 280. In embodiments, the polymerase includes arginine at amino acid position 280 or an amino acid position corresponding to position 280. In embodiments, the polymerase includes histidine at amino acid position 280 or an amino acid position corresponding to position 280. In embodiments, the polymerase includes glutamic acid at amino acid position 280 or an amino acid position corresponding to position 280. In embodiments, the polymerase includes aspartic acid at amino acid position 280 or an amino acid position corresponding to position 280. In embodiments, the polymerase includes glutamine at amino acid position 280 or an amino acid position corresponding to position 280. In embodiments, the polymerase includes asparagine at amino acid position 280 or an amino acid position corresponding to position 280. In embodiments, the polymerase includes alanine at amino acid position 280 or an amino acid position corresponding to position 280. In embodiments, the polymerase includes serine at amino acid position 280 or an amino acid position corresponding to position 280. In embodiments, the polymerase includes proline at amino acid position 280 or an amino acid position corresponding to position 280. In embodiments, the polymerase includes valine at amino acid position 280 or an amino acid position corresponding to position 280. In embodiments, the polymerase includes glycine at amino acid position 280 or an amino acid position corresponding to position 280.

In embodiments, the polymerase includes lysine at amino acid position 280. In embodiments, the polymerase includes arginine at amino acid position 280. In embodiments, the polymerase includes histidine at amino acid position 280. In embodiments, the polymerase includes glutamic acid at amino acid position 280. In embodiments, the polymerase includes aspartic acid at amino acid position 280. In embodiments, the polymerase includes glutamine at amino acid position 280. In embodiments, the polymerase includes asparagine at amino acid position 280. In embodiments, the polymerase includes alanine at amino acid position 280. In embodiments, the polymerase includes serine at amino acid position 280. In embodiments, the polymerase includes proline at amino acid position 280. In embodiments, the polymerase includes valine at amino acid position 280. In embodiments, the polymerase includes glycine at amino acid position 280.

In embodiments, the polymerase includes methionine, alanine, serine, leucine, isoleucine, valine, or cysteine at amino acid position 241 or an amino acid position corresponding to position 241. In embodiments, the polymerase includes methionine at amino acid position 241 or an amino acid position corresponding to position 241. In embodiments, the polymerase includes alanine at amino acid position 241 or an amino acid position corresponding to position 241. In embodiments, the polymerase includes serine at amino acid position 241 or an amino acid position corresponding to position 241. In embodiments, the polymerase includes leucine at amino acid position 241 or an amino acid position corresponding to position 241. In embodiments, the polymerase includes isoleucine at amino acid position 241 or an amino acid position corresponding to position 241. In embodiments, the polymerase includes valine at amino acid position 241 or an amino acid position corresponding to position 241. In embodiments, the polymerase includes cysteine at amino acid position 241 or an amino acid position corresponding to position 241.

In embodiments, the polymerase includes methionine at amino acid position 241. In embodiments, the polymerase includes alanine at amino acid position 241. In embodiments, the polymerase includes serine at amino acid position 241. In embodiments, the polymerase includes leucine at amino acid position 241. In embodiments, the polymerase includes isoleucine at amino acid position 241. In embodiments, the polymerase includes valine at amino acid position 241. In embodiments, the polymerase includes cysteine at amino acid position 241.

In embodiments, the polymerase includes asparagine, lysine, aspartic acid, glutamine, serine, threonine, tyrosine, or glutamic acid at amino acid position 236 or an amino acid position corresponding to position 236. In embodiments, the polymerase includes asparagine at amino acid position 236 or an amino acid position corresponding to position 236. In embodiments, the polymerase includes lysine at amino acid position 236 or an amino acid position corresponding to position 236. In embodiments, the polymerase includes aspartic acid at amino acid position 236 or an amino acid position corresponding to position 236. In embodiments, the polymerase includes glutamine at amino acid position 236 or an amino acid position corresponding to position 236. In embodiments, the polymerase includes serine at amino acid position 236 or an amino acid position corresponding to position 236. In embodiments, the polymerase includes threonine at amino acid position 236 or an amino acid position corresponding to position 236. In embodiments, the polymerase includes tyrosine at amino acid position 236 or an amino acid position corresponding to position 236. In embodiments, the polymerase includes glutamic acid at amino acid position 236 or an amino acid position corresponding to position 236.

In embodiments, the polymerase includes asparagine at amino acid position 236. In embodiments, the polymerase includes lysine at amino acid position 236. In embodiments, the polymerase includes aspartic acid at amino acid position 236. In embodiments, the polymerase includes glutamine at amino acid position 236. In embodiments, the polymerase includes serine at amino acid position 236. In embodiments, the polymerase includes threonine at amino acid position 236. In embodiments, the polymerase includes tyrosine at amino acid position 236. In embodiments, the polymerase includes glutamic acid at amino acid position 236.

In embodiments, the polymerase includes a glutamine, valine, arginine, or alanine at amino acid position 93 or the amino acid position corresponding to position 93. In embodiments, the polymerase includes a glutamine at amino acid position 93 or the amino acid position corresponding to position 93. In embodiments, the polymerase includes a valine at amino acid position 93 or the amino acid position corresponding to position 93. In embodiments, the polymerase includes an arginine at amino acid position 93 or the amino acid position corresponding to position 93. In embodiments, the polymerase includes an alanine at amino acid position 93 or the amino acid position corresponding to position 93. In embodiments, the polymerase includes a glutamine at amino acid position 93. In embodiments, the polymerase includes a valine at amino acid position 93. In embodiments, the polymerase includes an arginine at amino acid position 93. In embodiments, the polymerase includes an alanine at amino acid position 93.

It is known that the presence of uracil in DNA results in a dramatic increase in the binding affinity of archacal family B DNA polymerases, stalling further polymerase activity (Lasken R S et al. J. Biol. Chem. 1996, 271 (30):17692-6 and Fogg M J et al. Nature Structural Biology. 2002, 9:922-7). A specific point mutation in the uracil-binding pocket of these polymerases disrupts uracil binding and allows extension in the presence of uracil without compromising polymerase activity (Norholm MH BMC Biotechnology. 2010, 10:21). Provided herein are novel DNA polymerase variants (e.g., V93Q, V93R, V93A) that disrupt the uracil binding pocket. In embodiments, the polymerase includes a V93Q, V93R, or V93A mutation. In embodiments, the polymerase includes a V93Q mutation. In embodiments, the polymerase includes a V93I, V93L, V93N, V93D, or V93E mutation. In embodiments, the polymerase includes an amino acid substitution at position 93. In embodiments, the amino acid substitution at position 93 is a glutamine substitution. In embodiments, the amino acid substitution at position 93 is an arginine substitution. In embodiments, the amino acid substitution at position 93 is an alanine substitution. In embodiments, the amino acid substitution at position 93 is a leucine substitution. In embodiments, the amino acid substitution at position 93 is an isoleucine substitution.

In embodiments, the polymerase includes an alanine at amino acid position 141 or the amino acid position corresponding to position 141; and an alanine at amino acid position 143 or the amino acid position corresponding to position 143. In embodiments, the polymerase includes an alanine at amino acid position 141; and an alanine at amino acid position 143.

In embodiments, the polymerase includes an amino acid substitution at position 141. In embodiments, the amino acid substitution at position 141 is an alanine substitution. In embodiments, the amino acid substitution at position 141 is a glycine substitution.

In embodiments, the polymerase includes an amino acid substitution at position 143. In embodiments, the amino acid substitution at position 143 is an alanine substitution. In embodiments, the amino acid substitution at position 143 is a glycine, alanine, threonine, or serine substitution.

In embodiments, the polymerase includes an alanine at amino acid position 129 or an amino acid position corresponding to position 129. In embodiments, the polymerase includes a methionine at amino acid position 129 or an amino acid position corresponding to position 129. In embodiments, the polymerase includes an alanine at amino acid position 129. In embodiments, the polymerase includes a methionine at amino acid position 129.

In embodiments, the polymerase includes a serine at amino acid position 429 or an amino acid position corresponding to position 429; a serine at amino acid position 443 or an amino acid position corresponding to position 443; a serine at amino acid position 507 or an amino acid position corresponding to position 507; or a serine at amino acid position 510 or an amino acid position corresponding to position 510. In embodiments, the polymerase includes a serine at amino acid position 429 or an amino acid position corresponding to position 429. In embodiments, the polymerase includes a serine at amino acid position 443 or an amino acid position corresponding to position 443. In embodiments, the polymerase includes a serine at amino acid position 507 or an amino acid position corresponding to position 507. In embodiments, the polymerase includes a serine at amino acid position 510 or an amino acid position corresponding to position 510. In embodiments, the polymerase includes a serine at amino acid position 429. In embodiments, the polymerase includes a serine at amino acid position 443. In embodiments, the polymerase includes a serine at amino acid position 507. In embodiments, the polymerase includes a serine at amino acid position 510.

In embodiments, the polymerase includes an amino acid substitution at position 429. The amino acid substitution at position 429 may be a serine, glycine, threonine, asparagine, or alanine substitution. The amino acid substitution at position 429 may be a serine substitution. In embodiments, the substitution at position 429 includes a polar amino acid (e.g., threonine, asparagine, or glutamine). In embodiments, the amino acid substitution at position 429 is a selenocysteine.

In embodiments, the polymerase includes an amino acid substitution at position 443. The amino acid substitution at position 443 may be a serine, glycine, threonine, asparagine, or alanine substitution. The amino acid substitution at position 443 may be a serine substitution. In embodiments, the substitution at position 443 includes a polar amino acid (e.g., threonine, asparagine, or glutamine). In embodiments, the amino acid substitution at position 443 is a selenocysteine.

In embodiments, the polymerase further includes an amino acid substitution mutation at positions 429 and 443. The amino acid substitutions at positions 429 and 443 may be serine substitutions.

In embodiments, the polymerase includes E306G, V341L, Y494F, E581G, and F588L. In embodiments, the polymerase includes an E306G mutation. In embodiments, the polymerase includes a V341L mutation. In embodiments, the polymerase includes a Y494F mutation. In embodiments, the polymerase includes an E581G mutation. In embodiments, the polymerase includes an F588L mutation.

In embodiments, the polymerase includes E280K, M241I, and N236D. In embodiments, the polymerase includes an E280K mutation. In embodiments, the polymerase includes an M241I mutation. In embodiments, the polymerase includes an N236D mutation.

In embodiments, the polymerase includes a mutation at amino acid position 409 or an amino acid position corresponding to position 409. In embodiments, the mutation at amino acid position 409 or the amino acid position corresponding to position 409 is alanine, glutamine, tyrosine, phenylalanine, isoleucine, valine, cysteine, serine, or histidine.

In embodiments, the polymerase includes an alanine or serine at amino acid position 409 or the amino acid position corresponding to position 409; a glycine at amino acid position 410 or an amino acid position corresponding to position 410; and a proline, valine, glycine, isoleucine, or serine at amino acid position 411 or an amino acid position corresponding to position 411. In embodiments, the polymerase includes an alanine or serine at amino acid position 409; a glycine at amino acid position 410; and a proline, valine, glycine, isoleucine, or serine at amino acid position 411. In embodiments, the polymerase includes an alanine at amino acid position 409 or the amino acid position corresponding to position 409. In embodiments, the polymerase includes an alanine at amino acid position 409. In embodiments, the polymerase includes a serine at amino acid position 409 or the amino acid position corresponding to position 409. In embodiments, the polymerase includes a serine at amino acid position 409. In embodiments, the polymerase includes a glycine at amino acid position 410 or the amino acid position corresponding to position 410. In embodiments, the polymerase includes a glycine at amino acid position 410. In embodiments, the polymerase includes a proline at amino acid position 411 or the amino acid position corresponding to position 411. In embodiments, the polymerase includes a proline at amino acid position 411. In embodiments, the polymerase includes a valine at amino acid position 411 or the amino acid position corresponding to position 411. In embodiments, the polymerase includes a valine at amino acid position 411. In embodiments, the polymerase includes a glycine at amino acid position 411 or the amino acid position corresponding to position 411. In embodiments, the polymerase includes a glycine at amino acid position 411. In embodiments, the polymerase includes an isoleucine at amino acid position 411 or the amino acid position corresponding to position 411. In embodiments, the polymerase includes an isoleucine at amino acid position 411. In embodiments, the polymerase includes a serine at amino acid position 411 or the amino acid position corresponding to position 411. In embodiments, the polymerase includes a serine at amino acid position 411. In embodiments, the polymerase includes an alanine at amino acid position 409 or the amino acid position corresponding to 409; a glycine at amino acid position 410 or the amino acid position corresponding to 410; and a proline at amino acid position 411 or the amino acid position corresponding to 411. In embodiments, the polymerase includes an alanine at amino acid position 409; a glycine at amino acid position 410; and a proline at amino acid position 411.

In embodiments, the polymerase includes an alanine or serine at amino acid position 409 or the amino acid position corresponding to position 409; a glycine at amino acid position 410 or an amino acid position corresponding to position 410; and a proline, valine, glycine, isoleucine, or serine at amino acid position 411 or an amino acid position corresponding to position 411.

In embodiments, the first mutation at amino acid position 409 or the amino acid position corresponding to position 409 is alanine, glutamine, tyrosine, phenylalanine, isoleucine, valine, cysteine, serine, or histidine. In embodiments, the first mutation at amino acid position 409 is alanine, glutamine, tyrosine, phenylalanine, isoleucine, valine, cysteine, serine, or histidine. In embodiments, the first mutation at amino acid position 409 or the amino acid position corresponding to position 409 is alanine. In embodiments, the first mutation at amino acid position 409 is alanine. In embodiments, the first mutation at amino acid position 409 or the amino acid position corresponding to position 409 is glutamine. In embodiments, the first mutation at amino acid position 409 is glutamine. In embodiments, the first mutation at amino acid position 409 or the amino acid position corresponding to position 409 is tyrosine. In embodiments, the first mutation at amino acid position 409 is tyrosine. In embodiments, the first mutation at amino acid position 409 or the amino acid position corresponding to position 409 is phenylalanine. In embodiments, the first mutation at amino acid position 409 is phenylalanine. In embodiments, the first mutation at amino acid position 409 or the amino acid position corresponding to position 409 is isoleucine. In embodiments, the first mutation at amino acid position 409 is isoleucine. In embodiments, the first mutation at amino acid position 409 or the amino acid position corresponding to position 409 is valine. In embodiments, the first mutation at amino acid position 409 is valine. In embodiments, the first mutation at amino acid position 409 or the amino acid position corresponding to position 409 is cysteine. In embodiments, the first mutation at amino acid position 409 is cysteine. In embodiments, the first mutation at amino acid position 409 or the amino acid position corresponding to position 409 is serine. In embodiments, the first mutation at amino acid position 409 is serine. In embodiments, the first mutation at amino acid position 409 or the amino acid position corresponding to position 409 is histidine. In embodiments, the first mutation at amino acid position 409 is histidine.

In embodiments, the polymerase includes a glycine or alanine at amino acid position 410 or an amino acid position corresponding to position 410. In embodiments, the polymerase includes a glycine or alanine at amino acid position 410. In embodiments, the polymerase includes a glycine at amino acid position 410 or an amino acid position corresponding to position 410. In embodiments, the polymerase includes a glycine at amino acid position 410. In embodiments, the polymerase includes an alanine at amino acid position 410 or an amino acid position corresponding to position 410. In embodiments, the polymerase includes an alanine at amino acid position 410.

In embodiments, the polymerase includes a proline, serine, alanine, glycine, valine, or isoleucine at amino acid position 411 or an amino acid position corresponding to position 411. In embodiments, the polymerase includes a proline, serine, alanine, glycine, valine, or isoleucine at amino acid position 411. In embodiments, the polymerase includes a proline at amino acid position 411 or an amino acid position corresponding to position 411. In embodiments, the polymerase includes a proline at amino acid position 411. In embodiments, the polymerase includes a serine at amino acid position 411 or an amino acid position corresponding to position 411. In embodiments, the polymerase includes a serine at amino acid position 411. In embodiments, the polymerase includes an alanine at amino acid position 411 or an amino acid position corresponding to position 411. In embodiments, the polymerase includes an alanine at amino acid position 41. In embodiments, the polymerase includes a glycine at amino acid position 411 or an amino acid position corresponding to position 411. In embodiments, the polymerase includes a glycine at amino acid position 411. In embodiments, the polymerase includes a valine at amino acid position 411 or an amino acid position corresponding to position 411. In embodiments, the polymerase includes a valine at amino acid position 41. In embodiments, the polymerase includes an isoleucine at amino acid position 411 or an amino acid position corresponding to position 411. In embodiments, the polymerase includes an isoleucine at amino acid position 411.

In embodiments, the polymerase includes an amino acid substitution at position 409. The amino acid substitution at position 409 may be a serine substitution or an alanine substitution. In embodiments, the amino acid substitution at position 409 is a serine substitution. In embodiments, the amino acid substitution at position 409 is an alanine substitution. The amino acid substitution at position 409 may be a serine, cysteine, alanine, glycine, valine, isoleucine, glutamine, or histidine substitution. The amino acid substitution at position 409 may be a alanine, glycine, valine, isoleucine, threonine, glutamine, or histidine substitution.

In embodiments, the polymerase includes an amino acid substitution at position 410. The amino acid substitution at position 410 may be a glycine substitution or an alanine substitution. In embodiments, the amino acid substitution at position 410 is a glycine substitution. In embodiments, the amino acid substitution at position 410 is an alanine substitution. In embodiments, the amino acid substitution at position 410 is a valine substitution. In embodiments, the amino acid substitution at position 410 is a serine substitution. In embodiments, the amino acid substitution at position 410 is a proline substitution.

In embodiments, the polymerase includes an amino acid substitution at position 411. The amino acid substitution at position 411 may be an isoleucine substitution, a proline, a glycine substitution, a valine substitution, or a serine substitution. In embodiments, the amino acid substitution at position 411 is an isoleucine substitution. In embodiments, the amino acid substitution at position 411 is a proline. In embodiments, the amino acid substitution at position 411 is a glycine substitution. In embodiments, the amino acid substitution at position 411 is a valine substitution. In embodiments, the amino acid substitution at position 411 is a serine substitution. The amino acid substitution at position 411 may be glycine, alanine, leucine, isoleucine, proline, valine, leucine, serine, or threonine substitution. In embodiments, the amino acid substitution is a proline, alanine, or valine.

In embodiments, the polymerase does not comprise the following mutations: (L409S); (L409Q); (L409Y); or (L409F); (Y410G); (Y410A); or (Y410S); and (P411S); (P411I); (P411C); (P411A). In embodiments, the polymerase does not comprise L409S; Y410G; and P411I. In embodiments, the polymerase does not comprise L409S; Y410A; and P411I. In embodiments, the polymerase does not comprise L409S; Y410G; and P411S. In embodiments, the polymerase does not comprise L409S; Y410A; and P411S. In embodiments, the polymerase is not a wild type enzyme. In embodiments, the polymerase is a synthetic polymerase.

Functionally equivalent, positionally equivalent and homologous amino acids within the wild type amino acid sequences of two different polymerases do not necessarily have to be the same type of amino acid residue, although functionally equivalent, positionally equivalent and homologous amino acids are commonly conserved. By way of example, the motif A region of 9°N polymerase has the sequence LYP, the functionally homologous region of Vent™ polymerase also has sequence LYP. In the case of these two polymerases the homologous amino acid sequences are identical, however homologous regions in other polymerases may have different amino acid sequence. In embodiments, when describing an amino acid functionally equivalent to amino acid position 409, or describing an amino acid position functionally equivalent to amino acid position 409, positional equivalence and/or functional equivalence is referring to amino acid position 409 of SEQ ID NO: 1 or an amino acid at a position in a polymerase at least 80% identical to a continuous 500 amino acid sequence within SEQ ID NO: 1 that is equivalent to position 409 of SEQ ID NO:1. A person having ordinary skill in the art would recognize a positional equivalent of amino acid position 409 by performing a sequence alignment given that the polymerase must be at least 80% identical to a continuous 500 amino acid sequence within SEQ ID NO: 1.

In embodiments, the polymerase is selected from a Pyrococcus abyssi, Pyrococcus endeavori, Pyrococcus furiosus, Pyrococcus glycovorans, Pyrococcus horikoshii, Pyrococcus kukulkanii, Pyrococcus woesei, Pyrococcus yayanosii, Pyrococcus sp., Pyrococcus sp. 12/1, Pyrococcus sp. 121, Pyrococcus sp. 303, Pyrococcus sp. 304, Pyrococcus sp. 312, Pyrococcus sp. 32-4, Pyrococcus sp. 321, Pyrococcus sp. 322, Pyrococcus sp. 323, Pyrococcus sp. 324, Pyrococcus sp. 95-12-1, Pyrococcus sp. AV5, Pyrococcus sp. Ax99-7, Pyrococcus sp. C2, Pyrococcus sp. EX2, Pyrococcus sp. Fla95-Pc, Pyrococcus sp. GB-3A, Pyrococcus sp. GB-D, Pyrococcus sp. GBD, Pyrococcus sp. GI-H, Pyrococcus sp. GI-J, Pyrococcus sp. GIL, Pyrococcus sp. HT3, Pyrococcus sp. JTI, Pyrococcus sp. LMO-A29, Pyrococcus sp. LMO-A30, Pyrococcus sp. LMO-A31, Pyrococcus sp. LMO-A32, Pyrococcus sp. LMO-A33, Pyrococcus sp. LMO-A34, Pyrococcus sp. LMO-A35, Pyrococcus sp. LMO-A36, Pyrococcus sp. LMO-A37, Pyrococcus sp. LMO-A38, Pyrococcus sp. LMO-A39, Pyrococcus sp. LMO-A40, Pyrococcus sp. LMO-A41, Pyrococcus sp. LMO-A42, Pyrococcus sp. M24D13, Pyrococcus sp. MA2.31, Pyrococcus sp. MA2.32, Pyrococcus sp. MA2.34, Pyrococcus sp. MV1019, Pyrococcus sp. MV4, Pyrococcus sp. MV7, Pyrococcus sp. MZ14, Pyrococcus sp. MZ4, Pyrococcus sp. NA2, Pyrococcus sp. NS102-T, Pyrococcus sp. P12.1, Pyrococcus sp. Pikanate 5017, Pyrococcus sp. PK 5017, Pyrococcus sp. ST04, Pyrococcus sp. Tc-2-70, Pyrococcus sp. Tc95-7C-I, Pyrococcus sp. TC95-7C-S, Pyrococcus sp. Tc95_6, Pyrococcus sp. V211, Pyrococcus sp. V212, Pyrococcus sp. V221, Pyrococcus sp. V222, Pyrococcus sp. V231, Pyrococcus sp. V232, Pyrococcus sp. V61, Pyrococcus sp. V62, Pyrococcus sp. V63, Pyrococcus sp. V72, Pyrococcus sp. V73, Pyrococcus sp. VB112, Pyrococcus sp. VB113, Pyrococcus sp. VB81, Pyrococcus sp. VB82, Pyrococcus sp. VB83, Pyrococcus sp. VB85, Pyrococcus sp. VB86, Pyrococcus sp. VB93 polymerase, Pyrococcus furiosus DSM 3638, Pyrococcus sp. GE23, Pyrococcus sp. GI-H, Pyrococcus sp. NA2, Pyrococcus sp. ST04, or Pyrococcus sp. ST700 polymerase. In embodiments, the variants of a Pyrococcus family B DNA polymerase provided herein are a Pyrococcus horikoshii family B DNA polymerase that have strand-displacing activity and are useful in methods of incorporating modified nucleotides in nucleic acid synthesis reactions. In embodiments, the variants of a Pyrococcus family B DNA polymerase provided herein are a Pyrococcus abyssi family B DNA polymerase that have strand-displacing activity and are useful in methods of incorporating modified nucleotides in nucleic acid synthesis reactions.

In embodiments, the polymerase (e.g., a polymerase as described herein) is truncated at the C-terminus. In embodiments, the polymerase (e.g., a polymerase as described herein) is truncated at the C-terminus and retains the ability to incorporate a modified nucleotide. In embodiments, the polymerase (e.g., a polymerase as described herein) is truncated at the C-terminus, wherein the polymerase is truncated to remove at least 20 amino acids from the C-terminus. In embodiments, the polymerase (e.g., a polymerase as described herein) is truncated at the C-terminus, wherein the polymerase is truncated to remove at least 10 amino acids from the C-terminus. In embodiments, the polymerase (e.g., a polymerase as described herein) is truncated at the C-terminus, wherein the polymerase is truncated to remove at least 5 amino acids from the C-terminus. In embodiments, the polymerase (e.g., a polymerase as described herein) is truncated at the C-terminus, wherein the truncation removes 5 to 16 amino acids from the C-terminus. In embodiments, the polymerase (e.g., a polymerase as described herein) is truncated at the C-terminus, wherein the truncation removes 5 amino acids from the C-terminus. In embodiments, the polymerase (e.g., a polymerase as described herein) is truncated at the C-terminus, wherein the truncation removes 10 amino acids from the C-terminus. In embodiments, the polymerase (e.g., a polymerase as described herein) is truncated at the C-terminus, wherein the truncation removes 13 amino acids from the C-terminus. In embodiments, the polymerase (e.g., a polymerase as described herein) is truncated at the C-terminus, wherein the truncation removes 16 amino acids from the C-terminus.

In embodiments, the polymerase includes a polycationic sequence (e.g., a polyhistidine tag, such as a His-6 tag). To facilitate synthesis and/or purification, in embodiments a His₆ tag (i.e., six consecutive histidine amino acids) are ligated to the C or N terminus of the polypeptide chain. It is understood that the presence of a His₆ tag enables the isolation of peptide or protein products directly from ligation reaction mixtures by Ni-NTA affinity column purification. For example, common polyhistidine tags are formed of six histidine (6×His tag) residues which are added at the N-terminus preceded by methionine or C-terminus before a stop codon. Alternative polycationic sequences include alternating histidine and glutamine (e.g., three sets of HQ, referred to as an HQ tag) or alternating histidine and asparagine (e.g., six sets of HN, referred to as an HN tag). In embodiments, a 6×His-tag is attached to the C-terminus of the polymerase as described herein. In general, purification tags may be added to the polymerase (recombinantly or chemically) and include, e.g., polyhistidine tags, His₆-tags, biotin, avidin, GST sequences, BTag sequences, S tags, SNAP-tags, enterokinase sites, thrombin sites, antibodies or antibody domains, antibody fragments, antigens, receptors, receptor domains, and/or receptor fragments.

In embodiments, the polymerase is covalently attached to the polynucleotide-binding polypeptide. In embodiments, the polymerase is covalently attached to a Sso7d polypeptide. In embodiments, the polymerase is covalently attached to a Sac7d polypeptide. In embodiments, the polymerase is covalently attached to a Sac7e polypeptide. In embodiments, the polymerase is covalently attached to a Msc7 polypeptide. In embodiments, the polymerase is covalently attached to a Mcu7 polypeptide. In embodiments, the polymerase is covalently attached to a Aho7a polypeptide. In embodiments, the polymerase is covalently attached to a Aho7b polypeptide. In embodiments, the polymerase is covalently attached to a Aho7c polypeptide. In embodiments, the polymerase is covalently attached to a Sto7 polypeptide. In embodiments, the polymerase is covalently attached to a Ssh7b polypeptide. In embodiments, the polymerase is covalently attached to a Sis7a polypeptide. In embodiments, the polymerase is covalently attached to a Sis7b polypeptide. In embodiments, the polymerase is covalently attached to a Ssh7a polypeptide. In embodiments, the polymerase is covalently attached to a nucleoid-associated protein HU-alpha polypeptide. In embodiments, the polymerase is covalently attached to a Sso7d polypeptide at the N-terminal of the polymerase (e.g., SEQ ID NO: 1). In embodiments, the polymerase includes a linker between the polymerase and the polynucleotide-binding polypeptide. In embodiments, the polymerase is covalently attached to a Sso7d polypeptide at the C-terminal of the polymerase (e.g., SEQ ID NO:1).

In embodiments, the polynucleotide-binding polypeptide is isolated from Saccharolobus solfataricus. In embodiments, the polynucleotide-binding polypeptide is isolated from Sulfolobus acidocaldarius. In embodiments, the polynucleotide-binding polypeptide is isolated from Metallosphaera sedula. In embodiments, the polynucleotide-binding polypeptide is isolated from Metallosphaera cuprina. In embodiments, the polynucleotide-binding polypeptide is isolated from Acidianus hospitalis. In embodiments, the polynucleotide-binding polypeptide is isolated from Sulfurisphaera tokodaii. In embodiments, the polynucleotide-binding polypeptide is isolated from Sulfolobus islandicus. In embodiments, the polynucleotide-binding polypeptide is isolated from Saccharolobus shibatae.

In embodiments, the polynucleotide-binding polypeptide is a Sso7d polypeptide. In embodiments, the polynucleotide-binding polypeptide is a Sac7d polypeptide. In embodiments, the polynucleotide-binding polypeptide is a Sac7e polypeptide. In embodiments, the polynucleotide-binding polypeptide is a Msc7 polypeptide. In embodiments, the polynucleotide-binding polypeptide is a Mcu7 polypeptide. In embodiments, the polynucleotide-binding polypeptide is a Aho7a polypeptide. In embodiments, the polynucleotide-binding polypeptide is a Aho7b polypeptide. In embodiments, the polynucleotide-binding polypeptide is a Aho7c polypeptide. In embodiments, the polynucleotide-binding polypeptide is a Sto7 polypeptide. In embodiments, the polynucleotide-binding polypeptide is a Ssh7b polypeptide. In embodiments, the polynucleotide-binding polypeptide is a Sis7a polypeptide. In embodiments, the polynucleotide-binding polypeptide is a Sis7b polypeptide. In embodiments, the polynucleotide-binding polypeptide is a Ssh7a polypeptide. In embodiments, the polynucleotide-binding polypeptide is a nucleoid-associated protein HU-alpha polypeptide.

In embodiments, the composition includes a plurality of native DNA nucleotides including a plurality of dATP (2′-deoxyadenosine-5′-triphosphate) nucleotides, dCTP (2′-deoxycytidine-5′-triphosphate) nucleotides, dTTP (2′-deoxythymidine-5′-triphosphate) nucleotides, and dGTP (2′-deoxyguanosine-5′-triphosphate) nucleotides. In embodiments, the composition includes a plurality of dATP (2′-deoxyadenosine-5′-triphosphate) nucleotides, dCTP (2′-deoxycytidine-5′-triphosphate) nucleotides, dTTP (2′-deoxythymidine-5′-triphosphate) nucleotides, and dGTP (2′-deoxyguanosine-5′-triphosphate) nucleotides. In embodiments, the composition includes a plurality of native DNA nucleotides including a plurality of dATP nucleotides, dCTP nucleotides, dTTP nucleotides, or dGTP nucleotides. In embodiments, the composition includes a plurality of dATP nucleotides, dCTP nucleotides, dTTP nucleotides, or dGTP nucleotides. In embodiments, the composition includes a plurality of dATP nucleotides. In embodiments, the composition includes a plurality of dCTP nucleotides. In embodiments, the composition includes a plurality of dTTP nucleotides. In embodiments, the composition includes a plurality of dGTP nucleotides. In embodiments, the composition includes a plurality of dUTP (2′-deoxycytidine-5′-triphosphate) nucleotides. In embodiments, the composition consists of a plurality of dA nucleotides, a plurality of dC nucleotides, a plurality of dT nucleotides, and a plurality of dG nucleotides. In embodiments, the composition consists of a plurality of dA nucleotides, a plurality of dC nucleotides, a plurality of dT nucleotides, a plurality of dU nucleotides, and a plurality of dG nucleotides.

In embodiments, the composition includes a plurality of native RNA nucleotides (i.e., native ribonucleotides) including a plurality of ATP (adenosine-5′-triphosphate) nucleotides, CTP (cytidine-5′-triphosphate) nucleotides, UTP (uridine-5′-triphosphate) nucleotides, and GTP (guanosine-5′-triphosphate) nucleotides. In embodiments, the composition includes a plurality of native RNA nucleotides including a plurality of ATP nucleotides, CTP nucleotides, UTP nucleotides, or GTP nucleotides. In embodiments, the composition includes a plurality of ATP nucleotides. In embodiments, the composition includes a plurality of CTP nucleotides. In embodiments, the composition includes a plurality of UTP nucleotides. In embodiments, the composition includes a plurality of GTP nucleotides. In embodiments, the composition consists of a plurality of A ribonucleotides, a plurality of C ribonucleotides, a plurality of U ribonucleotides, and a plurality of G ribonucleotides.

In an aspect is provided a kit. In embodiments, the kit includes a polymerase as described herein. In embodiments, the kit includes the reagents and containers useful for performing the methods as described herein. Generally, the kit includes one or more containers providing a composition and one or more additional reagents (e.g., a buffer suitable for polynucleotide extension). The kit may also include a template nucleic acid (DNA and/or RNA), one or more primer polynucleotides, nucleoside triphosphates (including, for example, deoxyribonucleotides, ribonucleotides, and/or modified nucleotides), buffers, salts, and/or labels (e.g., fluorophores).

In embodiments, the kit includes a solid support (i.e., a substrate), and reagents for sample preparation and purification, amplification, and/or sequencing (e.g., one or more sequencing reaction mixtures). In embodiments, amplification reagents and other reagents may be provided in lyophilized form. In embodiments, amplification reagents and other reagents may be provided in a container which the lyophilized reagent may be reconstituted.

In embodiments, the kit includes components useful for circularizing template polynucleotides using a ligation enzyme (e.g., CircLigase™ enzyme, Taq DNA Ligase, HiFi Taq DNA Ligase, T4 ligase, SplintR® ligase, or Ampligase® DNA Ligase). For example, such a kit further includes the following components: (a) reaction buffer for controlling pH and providing an optimized salt composition for a ligation enzyme (e.g., CircLigase™ enzyme, Taq DNA Ligase, HiFi Taq DNA Ligase, T4 ligase, SplintR® ligase, or Ampligase® DNA Ligase), and (b) ligation enzyme cofactors. In embodiments, the kit further includes instructions for use thereof. CircLigase™ and Ampligase® are trademarks of Epicentre. SplintR® is a registered trademark of NEB.

In embodiments, kits described herein include a polymerase. In embodiments, the polymerase is a DNA polymerase. In embodiments, the kit includes a strand-displacing polymerase. In embodiments, the polymerase is a DNA polymerase. In embodiments, the DNA polymerase is a thermophilic nucleic acid polymerase. In embodiments, the DNA polymerase is a modified archacal DNA polymerase. In embodiments, the kit includes a strand-displacing polymerase, such as a polymerase as described herein.

In embodiments, the kit includes a sequencing solution, hybridization solution, and/or extension solution. In embodiments, the sequencing solution includes labeled nucleotides including differently labeled nucleotides, wherein the label (or lack thereof) identifies the type of nucleotide. For example, each adenine nucleotide, or analog thereof; a thymine nucleotide; a cytosine nucleotide, or analog thereof; and a guanine nucleotide, or analog thereof may be labeled with a different fluorescent label.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid. In embodiments, the buffered solution can contain one or more divalent cations at a concentration sufficient to permit hybridization of a nucleic acid. In some embodiments, a concentration can be more than about 1 μM, more than about 2 μM, more than about 5 μM, more than about 10 μM, more than about 25 μM, more than about 50 μM, more than about 75 μM, more than about 100 μM, more than about 200 μM, more than about 300 μM, more than about 400 μM, more than about 500 μM, more than about 750 μM, more than about 1 mM, more than about 2 mM, more than about 5 mM, more than about 10 mM, more than about 20 mM, more than about 30 mM, more than about 40 mM, more than about 50 mM, more than about 60 mM, more than about 70 mM, more than about 80 mM, more than about 90 mM, more than about 100 mM, more than about 150 mM, more than about 200 mM, more than about 250 mM, more than about 300 mM, more than about 350 mM, more than about 400 mM, more than about 450 mM, more than about 500 mM, more than about 550 mM, more than about 600 mM, more than about 650 mM, more than about 700 mM, more than about 750 mM, more than about 800 mM, more than about 850 mM, more than about 900 mM, more than about 950 mM or more than about 1 M. In embodiments, the buffered solution includes about 10 mM Tris, about 20 mM Tris, about 30 mM Tris, about 40 mM Tris, or about 50 mM Tris. In embodiments the buffered solution includes about 50 mM NaCl, about 75 mM NaCl, about 100 mM NaCl, about 125 mM NaCl, about 150 mM NaCl, about 200 mM NaCl, about 300 mM NaCl, about 400 mM NaCl, or about 500 mM NaCl. In embodiments, the buffered solution includes about 0.05 mM EDTA, about 0.1 mM EDTA, about 0.25 mM EDTA, about 0.5 mM EDTA, about 1.0 mM EDTA, about 1.5 mM EDTA or about 2.0 mM EDTA. In embodiments, the buffered solution includes about 0.01% Triton™ X-100, about 0.025% Triton™ X-100, about 0.05% Triton™ X-100, about 0.1% Triton™ X-100, or about 0.5% Triton™ X-100. In embodiments, the buffered solution includes 20 mM Tris pH 8.0, 100 mM NaCl, 0.1 mM EDTA, 0.025% Triton™ X-100. In embodiments, the buffered solution includes 20 mM Tris pH 8.0, 150 mM NaCl, 0.1 mM EDTA, 0.025% Triton™ X-100. In embodiments, the buffered solution includes 20 mM Tris pH 8.0, 300 mM NaCl, 0.1 mM EDTA, 0.025% Triton™ X-100. In embodiments, the buffered solution includes 20 mM Tris pH 8.0, 400 mM NaCl, 0.1 mM EDTA, 0.025% Triton™ X-100. In embodiments, the buffered solution includes 20 mM Tris pH 8.0, 500 mM NaCl, 0.1 mM EDTA, 0.025% Triton™ X-100. Triton™ is a registered trademark of Dow Chemical Company.

In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, digital storage medium, etc., on which the information has been recorded. Yet another means that may be present is a website address which may be used via the Internet to access the information at a removed site. Any convenient means may be present in the kits.

### III. Methods

In an aspect, a method of incorporating, and optionally detecting, a modified nucleotide into a nucleic acid sequence is provided. The method includes allowing the following components to interact: (i) a nucleic acid template, (ii) a primer that has an extendible 3′ end, (iii) a nucleotide solution, and (iv) a polymerase (e.g., a DNA polymerase or a thermophilic nucleic acid polymerase as described herein). The polymerase used in the method includes an amino acid sequence that is at least 80% identical to a continuous 500 amino acid sequence within SEQ ID NO: 1 and includes one or more of the mutations described herein. In embodiments, the polymerase includes substitution mutations at positions 141 and 143 of SEQ ID NO: 1. In embodiments, the polymerase further includes at least one amino acid substitution mutation at a position selected from positions 409, 410, and 411 of SEQ ID NO: 1. In embodiments, the polymerase includes a mutation as described herein. In embodiments, the method includes incorporating the nucleotide into a nucleic acid molecule in a cell or tissue.

In an aspect is provided a method of incorporating a nucleotide into a nucleic acid sequence including combining in a reaction vessel: (i) a nucleic acid template, (ii) a nucleotide solution, and (iii) a polymerase, wherein the polymerase is a polymerase as described herein. In embodiments, the nucleic acid template is bound to a primer including the nucleic acid sequence. In embodiments, the nucleotide is a modified nucleotide. In embodiments, the modified nucleotide is incorporated into the primer.

In another aspect is provided method of sequencing a nucleic acid sequence including: a. hybridizing a nucleic acid template with a primer to form a primer-template hybridization complex; b. contacting the primer-template hybridization complex with a DNA polymerase and modified nucleotides, wherein the DNA polymerase is the polymerase of any one of claims 1 to 33, wherein the modified nucleotide includes a detectable label; c. incorporating a modified nucleotide into the primer-template hybridization complex with the DNA polymerase to form a modified primer-template hybridization complex; and d. detecting the detectable label; thereby sequencing a nucleic acid sequence.

In an aspect is provided a method of incorporating a modified nucleotide into a nucleic acid sequence including combining in a reaction vessel: (i) a nucleic acid template, (ii) a nucleotide solution, and (iii) a polymerase, wherein the polymerase is a polymerase as described herein. In embodiments, the modified nucleotide includes a label (e.g., a label linked to the nucleobase via an optionally cleavable linker). In embodiments, the modified nucleotide includes a reversible terminator moiety (e.g., a polymerase-compatible cleavable moiety bonded to the 3′ oxygen of a nucleotide). In embodiments, the method includes combining the components in a reaction vessel under conditions for incorporating and/or polymerization. Such conditions are known in the art and described herein.

In another aspect is provided a method of sequencing a nucleic acid sequence including: a. hybridizing a nucleic acid template with a primer to form a primer-template hybridization complex; b. contacting the primer-template hybridization complex with a DNA polymerase and modified nucleotides, wherein the DNA polymerase is the polymerase as described herein, wherein the modified nucleotide includes a detectable label; c. subjecting the primer-template hybridization complex to conditions which enable the polymerase to incorporate a modified nucleotide into the primer-template hybridization complex to form a modified primer-template hybridization complex; and d. detecting the detectable label; thereby sequencing a nucleic acid sequence.

In an aspect is provided a method of incorporating a nucleotide into a primed nucleic acid template (e.g., a primer hybridized to a template nucleic acid). In embodiments, the method includes combining in a reaction vessel: (i) a primer hybridized to a nucleic acid template, (ii) a nucleotide solution including a plurality of nucleotides, and (iii) a polymerase, wherein the polymerase is a polymerase as described herein.

In embodiments, the template polynucleotide includes genomic DNA, complementary DNA (cDNA), cell-free DNA (cfDNA), messenger RNA (mRNA), transfer RNA (IRNA), ribosomal RNA (rRNA), cell-free RNA (cfRNA), or noncoding RNA (ncRNA). In embodiments, the template polynucleotide includes double-stranded DNA. In embodiments, the method of forming the template polynucleotide includes ligating a hairpin adapter to an end of a linear polynucleotide. In embodiments, the method of forming the template polynucleotide includes ligating hairpin adapters to both ends of the linear polynucleotide. In embodiments, the method of forming the template polynucleotide includes ligating a Y-shaped adapter to an end of a linear polynucleotide. In embodiments, the method of forming the template polynucleotide includes ligating a Y-shaped adapter to both ends of a linear polynucleotide.

In embodiments, the template polynucleotide is about 100 to 1000 nucleotides in length. In embodiments, the template polynucleotide is about 350 nucleotides in length. In embodiments, the template polynucleotide is about 10, 20, 50, 100, 150, 200, 300, or 500 nucleotides in length. The template polynucleotide molecules can vary length, such as about 100-300 nucleotides long, about 300-500 nucleotides long, or about 500-1000 nucleotides long. In embodiments, the template polynucleotide molecular is about 100-1000 nucleotides, about 150-950 nucleotides, about 200-900 nucleotides, about 250-850 nucleotides, about 300-800 nucleotides, about 350-750 nucleotides, about 400-700 nucleotides, or about 450-650 nucleotides. In embodiments, the template polynucleotide molecule is about 150 nucleotides. In embodiments, the template polynucleotide is about 100-1000 nucleotides long. In embodiments, the template polynucleotide is about 100-300 nucleotides long. In embodiments, the template polynucleotide is about 300-500 nucleotides long. In embodiments, the template polynucleotide is about 500-1000 nucleotides long. In embodiments, the template polynucleotide molecule is about 100 nucleotides. In embodiments, the template polynucleotide molecule is about 300 nucleotides. In embodiments, the template polynucleotide molecule is about 500 nucleotides. In embodiments, the template polynucleotide molecule is about 1000 nucleotides.

In embodiments the template polynucleotide (e.g., genomic template DNA) is first treated to form single-stranded linear fragments (e.g., ranging in length from about 50 to about 600 nucleotides). Treatment typically entails fragmentation, such as by chemical fragmentation, enzymatic fragmentation, or mechanical fragmentation, followed by denaturation to produce single-stranded DNA fragments. In embodiments, the template polynucleotide includes an adapter. The adaptor may have other functional elements including tagging sequences (i.e., a barcode), attachment sequences, palindromic sequences, restriction sites, sequencing primer binding sites, functionalization sequences, and the like. Barcodes can be of any of a variety of lengths. In embodiments, the primer includes a barcode that is 10-50, 20-30, or 4-12 nucleotides in length. In embodiments, the adapter includes a primer binding sequence that is complementary to at least a portion of a primer (e.g., a sequencing primer). Primer binding sites can be of any suitable length. In embodiments, a primer binding site is about or at least about 10, 15, 20, 25, 30, or more nucleotides in length. In embodiments, a primer binding site is 10-50, 15-30, or 20-25 nucleotides in length.

In embodiments, the template polynucleotide is single-stranded DNA, double-stranded DNA, single-stranded RNA, or double-stranded RNA. In embodiments, the template is single-stranded DNA or single-stranded RNA and is about 10, 20, 50, 100, 150, 200, 300, 500, or 1000 nucleotides in length. In embodiments, the template polynucleotide is double-stranded DNA or double-stranded RNA and is about 10, 20, 50, 100, 150, 200, 300, 500, or 1000 base pairs in length. In embodiments, the template polynucleotide includes single-stranded circular DNA. In embodiments, the template polynucleotide is single-stranded circular DNA. In embodiments, the template polynucleotide includes double-stranded DNA. In embodiments, the template polynucleotide is double-stranded DNA. In embodiments, the template polynucleotide includes single-stranded RNA. In embodiments, the template polynucleotide is single-stranded RNA. In embodiments, the template polynucleotide includes double-stranded RNA. In embodiments, the template polynucleotide is double-stranded RNA. In embodiments, the template polynucleotide includes primer binding sequences that are complementary to one or more substrate-bound primers. In embodiments, the substrate-bound primers are immobilized to a substrate by a covalent linker. In embodiments, the substrate-bound primers are immobilized to a solid support at the 5′ end, preferably via a covalent attachment. In embodiments, the template polynucleotide includes primer binding sequences that are complementary to one or more immobilized primers. In embodiments, the immobilized primers are immobilized to a matrix (e.g., a matrix in a cell) by a covalent linker. In embodiments, the immobilized primers are attached to a matrix at the 5′ end, preferably via a covalent attachment. In embodiments, at least some of the substrate-bound primers are phosphorothioated primers. In embodiments, a fraction of the total of the substrate-bound primers are phosphorothioated primers. In embodiments, at least some of the immobilized primers are phosphorothioated primers. In embodiments, a fraction of the total of the immobilized primers are phosphorothioated primers.

In another aspect is provided a method of amplifying a nucleic acid sequence, the method including hybridizing a nucleic acid template to a primer to form a primer-template hybridization complex; contacting the primer-template hybridization complex with a DNA polymerase and a plurality of nucleotides, wherein the DNA polymerase is the polymerase is as described herein; and subjecting the primer-template hybridization complex to conditions which enable the polymerase to incorporate one or more nucleotides into the primer-template hybridization complex to generate amplification products, thereby amplifying a nucleic acid sequence.

In embodiments, the nucleic acid template is DNA, RNA, or analogs thereof. In embodiments, the nucleic acid template includes a primer hybridized to the template. In embodiments, the nucleic acid template is a primer. Primers are usually single-stranded for maximum efficiency in amplification, but may alternatively be double-stranded. If double-stranded, the primer is usually first treated to separate its strands before being used to prepare extension products. This denaturation step is typically affected by heat, but may alternatively be carried out using alkali, followed by neutralization. Thus, a “primer” is complementary to a nucleic acid template, and complexes by hydrogen bonding or hybridization with the template to give a primer/template complex for initiation of synthesis by a polymerase, which is extended by the addition of covalently bonded bases linked at their 3′ end complementary to the template in the process of DNA synthesis. The DNA template for a sequencing reaction will typically comprise a double-stranded region having a free 3′ hydroxyl group which serves as a primer or initiation point for the addition of further nucleotides in the sequencing reaction. The region of the DNA template to be sequenced will overhang this free 3′ hydroxyl group on the complementary strand. The primer bearing the free 3′ hydroxyl group may be added as a separate component (e.g. a short oligonucleotide), which hybridizes to a region of the template to be sequenced. Alternatively, the primer and the template strand to be sequenced may each form part of a partially self-complementary nucleic acid strand capable of forming an intramolecular duplex, such as for example a hairpin loop structure. Nucleotides are added successively to the free 3′ hydroxyl group, resulting in synthesis of a polynucleotide chain in the 5′ to 3′ direction. After each nucleotide addition the nature of the base which has been added will be determined, thus providing sequence information for the DNA template.

In embodiments, the primer is hybridized to a polynucleotide in suitable hybridization conditions (e.g., saline-sodium citrate (SSC) buffer (pH 7.0), which is commonly used in nucleic acid hybridization techniques at concentrations from 0.1× to 20×). For example, hybridization may occur in the presence of an hybridization solution as described herein. For example, the hybridization solution may include 40% (v/v) formamide, 5×SSC, 5×Denhardt's solution, 0.1% (w/v) SDS, and dextran sulfate. In embodiments, the hybridization solution includes a buffered solution including salts (e.g., NaCl or KCl), a surfactant (e.g., Triton™ X-100 or Tween®-20), and, optionally, a chelator. In embodiments, the hybridization solution has a pH of about 7.5, 8.0, 8.2, 8.4, 8.6, 8.8, or 9.0. In embodiments, the hybridization solution includes NaCl or KCl, Tris (e.g., pH 8.0), Triton™ X-100, and a chelator (e.g., EDTA). In embodiments, the hybridization solution includes NaCl, Tris (e.g., pH 8.5), Triton™ X-100, and a chelator (e.g., EDTA). In embodiments, the hybridization solution includes NaCl, Tris (e.g., pH 8.8), Triton™ X-100, and a chelator (e.g., EDTA). In embodiments, the hybridization solution includes NaCl, Tris (e.g., pH 8.5), Tween®-20, and a chelator (e.g., EDTA). In embodiments, the hybridization solution includes NaCl, Tris (e.g., pH 8.8), Tween®-20, and a chelator (e.g., EDTA). In embodiments, the hybridization solution includes 3 M NaCl, 0.1 M Tris-HCl (pH 6.8), 0.1 M NaPO₄ buffer (pH 6.8), and 50 mM EDTA. In embodiments, the hybridization solution includes formamide. In embodiments, the hybridization solution includes dextran sulfate. In embodiments, the hybridization solution includes 140 mM HEPES, pH 8.0, containing 1% SDS, 1.7 M NaCl, 7×Denhardt's solution, 0.2 mM EDTA, and 3% PEG. In embodiments, the hybridization solution includes acetonitrile at 25-50% by volume, formamide at 5-10% by volume; 2-(N-morpholino) ethanesulfonic acid (MES); and polyethylene glycol (PEG) at 5-35%. In some embodiments, the hybridization solution further includes betaine.

In embodiments, extending is performed in the presence of an extension solution. In embodiments, the extension solution includes a buffered solution including salts (e.g., NaCl or KCl), a surfactant (e.g., Triton™ X-100 or Tween®-20), and a chelator. In embodiments, the extension solution includes nucleotides and a polymerase (e.g., a polymerase as described herein). In embodiments, the polymerase is a strand-displacing polymerase as described herein. In embodiments, the extension solution includes about 0.5, about 1, about 2, about 3, about 4, about 5, about 6, about 7, about 8, about 9, about 10, about 11, about 12, about 13, about 14, or about 15 mM Mg²⁺. In embodiments, the extension solution includes a dNTP mixture including dATP, dCTP, dGTP and dTTP (for DNA amplification) or dATP, dCTP, dGTP and dUTP (for RNA amplification). In embodiments, the extension solution has a pH of about 7.5, 8.0, 8.2, 8.4, 8.6, 8.8, or 9.0. In embodiments, the extension solution includes Tris-HCl (e.g., pH 8.0), salt (e.g, NaCl or KCl), MgSO₄, a surfactant (e.g., Tween®-20 or Triton™ X-100), dNTPs, BstLF, betaine (e.g., between about 0 to about 3.5M betaine), and/or DMSO (e.g., between about 0% to about 12% DMSO). In embodiments, the extension solution includes bicine (e.g., pH 8.5), salt (e.g., NaCl or KCl), MgSO₄, a surfactant (e.g., Tween-20 or Triton X-100), dNTPs, BstLF, (e.g., between about 0 to about 3.5M betaine), and/or DMSO (e.g., between about 0% to about 12% DMSO).

In embodiments, the hybridization solution and/or the extension solution includes a buffer such as, phosphate buffered saline (PBS), succinate, citrate, histidine, acetate, Tris, TAPS, MOPS, PIPES, HEPES, MES, and the like. The choice of appropriate buffer will generally be dependent on the target pH of the hybridization solution and/or the extension solution. In general, the desired pH of the buffer solution will range from about pH 4 to about pH 8.4. In some embodiments, the buffer pH may be at least 4.0, at least 4.5, at least 5.0, at least 5.5, at least 6.0, at least 6.2, at least 6.4, at least 6.6, at least 6.8, at least 7.0, at least 7.2, at least 7.4, at least 7.6, at least 7.8, at least 8.0, at least 8.2, or at least 8.4. In some embodiments, the buffer pH may be at most 8.4, at most 8.2, at most 8.0, at most 7.8, at most 7.6, at most 7.4, at most 7.2, at most 7.0, at most 6.8, at most 6.6, at most 6.4, at most 6.2, at most 6.0, at most 5.5, at most 5.0, at most 4.5, or at most 4.0. Any of the lower and upper values described in this paragraph may be combined to form a range included within the present disclosure, for example, in some instances, the desired pH may range from about 6.4 to about 7.2. Those of skill in the art will recognize that the buffer pH may have any value within this range, for example, about 7.25.

Suitable detergents for use in the hybridization solution and/or the extension solution include, but are not limited to, zwitterionic detergents (e.g., 1-Dodecanoyl-sn-glycero-3-phosphocholine, 3-(4-tert-Butyl-1-pyridinio)-1-propanesulfonate, 3-(N,N-Dimethylmyristylammonio) propanesulfonate, 3-(N,NDimethylmyristylammonio) propanesulfonate, ASB-C80, C7BzO, CHAPS, CHAPS hydrate, CHAPSO, DDMAB, Dimethylethylammoniumpropane sulfonate, N,N-Dimethyldodecylamine Noxide, N-Dodecyl-N,N-dimethyl-3-ammonio-1-propanesulfonate, or N-Dodecyl-N,N-dimethyl-3-ammonio-1-propanesulfonate) and anionic, cationic, and non-ionic detergents. Examples of nonionic detergents include poly(oxyethylene) ethers and related polymers (e.g. Brij®, TWEEN®, TWEEN®-20, TRITON™, TRITON™ X-100 and IGEPAL® CA-630), bile salts, and glycosidic detergents. In embodiments, the hybridization solution and/or the extension solution include antioxidants and reducing agents, carbohydrates, BSA, polyethylene glycol, dextran sulfate, betaine, other additives.

In embodiments, the method includes rolling circle amplification (RCA). In embodiments, the method includes exponential rolling circle amplification (eRCA). Exponential RCA is similar to the linear process except that it uses a second primer having a sequence that is identical to at least a portion of the circular template (Lizardi et al. Nat. Genet. 19:225 (1998)). This two-primer system achieves isothermal, exponential amplification. Exponential RCA has been applied to the amplification of non-circular DNA through the use of a linear probe that binds at both of its ends to contiguous regions of a target DNA followed by circularization using DNA ligase (Nilsson et al. Science 265(5181):208 5(1994)).

In embodiments, the method includes amplifying a template nucleic acid by extending an amplification primer with a strand-displacing polymerase for about 10 seconds to about 30 minutes. In embodiments, the method includes amplifying a template nucleic acid by extending an amplification primer with a strand-displacing polymerase for about 30 seconds to about 16 minutes. In embodiments, the method includes amplifying a template nucleic acid by extending an amplification primer with a strand-displacing polymerase for about 30 seconds to about 10 minutes. In embodiments, the method includes amplifying a template nucleic acid by extending an amplification primer with a strand-displacing polymerase for about 30 seconds to about 5 minutes. In embodiments, the method includes amplifying a template nucleic acid by extending an amplification primer with a strand-displacing polymerase for about 1 second to about 5 minutes. In embodiments, the method includes amplifying a template nucleic acid by extending an amplification primer with a strand-displacing polymerase for about 1 second to about 2 minutes.

In embodiments, the method includes amplifying a template nucleic acid by extending an amplification primer with a strand-displacing polymerase at a temperature of about 20° C. to about 50° C. In embodiments, the method includes amplifying a template nucleic acid by extending an amplification primer with a strand-displacing polymerase at a temperature of about 30° C. to about 50° C. In embodiments, the method includes amplifying a template nucleic acid by extending an amplification primer with a strand-displacing polymerase at a temperature of about 25° C. to about 45° C. In embodiments, the method includes amplifying a template nucleic acid by extending an amplification primer with a strand-displacing polymerase at a temperature of about 35° C. to about 45° C. In embodiments, the method includes amplifying a template nucleic acid by extending an amplification primer with a strand-displacing polymerase at a temperature of about 35° C. to about 42° C. In embodiments, the method includes amplifying a template nucleic acid by extending an amplification primer with a strand-displacing polymerase at a temperature of about 37° C. to about 40° C.

In embodiments, the method further includes detecting the amplification products. In embodiments, detecting the amplification products includes detecting a label (e.g., a labeled oligonucleotide bound to an amplification product or a labeled nucleotide bound to a primer bound to the amplification product). In embodiments, detecting the amplification products includes detecting the label of a fluorescently labeled oligonucleotide. In embodiments, detecting includes sequencing. In embodiments, sequencing includes extending a sequencing primer annealed to the amplification product to incorporate a nucleotide containing a detectable label that indicates the identity of a nucleotide in the amplification product, detecting the detectable label, and optionally repeating the extending and detecting of steps. In embodiments, the methods include sequencing one or more bases of a target nucleic acid (e.g., amplification product) by extending a sequencing primer hybridized to the target nucleic acid (e.g., an amplification product of a target nucleic acid). In embodiments, the sequencing includes sequencing-by-synthesis, sequencing-by-binding, sequencing by ligation, sequencing-by-hybridization, or pyrosequencing, and generates a sequencing read. In embodiments, generating a sequencing read includes executing a plurality of sequencing cycles, each cycle including extending the sequencing primer by incorporating a nucleotide or nucleotide analogue using a polymerase and detecting a characteristic signature indicating that the nucleotide or nucleotide analogue has been incorporated.

In embodiments, the nucleotide solution includes modified nucleotides. It is understood that a modified nucleotide and a nucleotide analogue are interchangeable terminology in this context. In embodiments, the nucleotide solution includes labelled nucleotides. In embodiments, the nucleotides include synthetic nucleotides. In embodiments, the nucleotide solution includes modified nucleotides that independently have different reversible terminating moieties. In embodiments the nucleotide solution contains native nucleotides. In embodiments the nucleotide solution contains labelled nucleotides.

In embodiments, the modified nucleotide has a removable group, for example a label, a blocking group, or protecting group. The removable group includes a chemical group that can be removed from a dNTP analogue such that a DNA polymerase can extend the nucleic acid (e.g., a primer or extension product) by the incorporation of at least one additional nucleotide. In embodiments, the removal group is a reversible terminator.

In embodiments, the modified nucleotide includes a blocking moiety and/or a label moiety. The blocking moiety on a nucleotide can be reversible, whereby the blocking moiety can be removed or modified to allow the 3′ hydroxyl to form a covalent bond with the 5′ phosphate of another nucleotide. The blocking moiety can be effectively irreversible under particular conditions used in a method set forth herein. A label moiety of a nucleotide can be any moiety that allows the nucleotide to be detected, for example, using a spectroscopic method. In embodiments, one or more of the above moieties can be absent from a nucleotide used in the methods and compositions set forth herein. For example, a nucleotide can lack a label moiety or a blocking moiety or both.

In embodiments, the blocking moiety can be located, for example, at the 3′ position of the nucleotide and may be a chemically cleavable moiety such as an allyl group, an azidomethyl group or a methoxymethyl group, or may be an enzymatically cleavable group such as a phosphate. Suitable nucleotide blocking moieties are described in applications WO 2004/018497, U.S. Pat. Nos. 10,738,072, 7,057,026, 7,541,444, WO 96/07669, U.S. Pat. Nos. 5,763,594, 5,808,045, 5,872,244 and 6,232,465, the contents of which are incorporated herein by reference in their entirety. The nucleotides may be labelled or unlabeled. In embodiments, the modified nucleotides with reversible terminators useful in methods provided herein may be 3′-O-blocked reversible or 3′-unblocked reversible terminators. The 3′-O-blocked reversible terminators are known in the art, and may be, for instance, a 3′-ONH₂ reversible terminator, a 3′-O-allyl reversible terminator, or a 3′-O-azidomethyl reversible terminator.

In embodiments, the modified nucleotides useful in methods provided herein can include 3′-unblocked reversible terminators. The 3′-unblocked reversible terminators are known in the art and include for example, the “virtual terminator” as described in U.S. pat. No. 8,11,4973 and the “lightening terminator” as described in U.S. Pat. No. 10,041,115, the contents of which are incorporated herein by reference in their entirety.

In embodiments, the modified nucleotide (also referred to herein as a nucleotide analogue) has the formula:

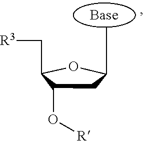

wherein Base is an optionally substituted nucleobase as described herein, R³ is-OH, monophosphate, or polyphosphate or a nucleic acid, and R′ is a reversible terminator. In embodiments, R′ has the formula:

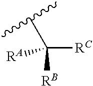

wherein R^{A} and R^{B} are hydrogen or alkyl and R^{C} is the remainder of the reversible terminator (e.g., an azido or SS—C₁-C₆ alkyl). In embodiments, the nucleotide is

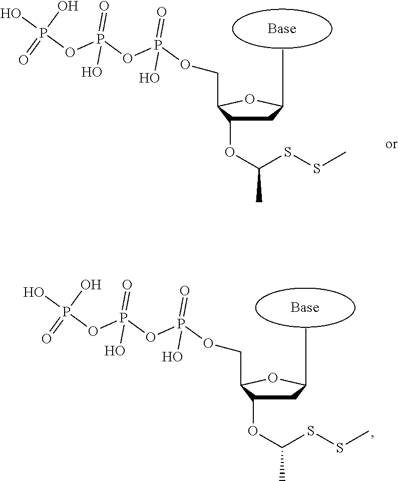

wherein the Base is cytosine or a derivative thereof (e.g., cytosine analogue), guanine or a derivative thereof (e.g., guanine analogue), adenine or a derivative thereof (e.g., adenine analogue), thymine or a derivative thereof (e.g., thymine analogue), uracil or a derivative thereof (e.g., uracil analogue), hypoxanthine or a derivative thereof (e.g., hypoxanthine analogue), xanthine or a derivative thereof (e.g., xanthine analogue), guanosine or a derivative thereof (e.g., 7-methylguanosine analogue), deaza-adenine or a derivative thereof (e.g., deaza-adenine analogue), deaza-guanine or a derivative thereof (e.g., deaza-guanine), deaza-hypoxanthine or a derivative thereof, 5,6-dihydrouracil or a derivative thereof (e.g., 5,6-dihydrouracil analogue), 5-methylcytosine or a derivative thereof (e.g., 5-methylcytosine analogue), or 5-hydroxymethylcytosine or a derivative thereof (e.g., 5-hydroxymethylcytosine analogue) moieties. In embodiments, the base is thymine, cytosine, uracil, adenine, guanine, hypoxanthine, xanthine, theobromine, caffeine, uric acid, or isoguanine.

In embodiments, mutations may include substitution of the amino acid in the parent amino acid sequences with an amino acid, which is not the parent amino acid. In embodiments, the mutations may result in conservative amino acid changes. In embodiments, non-polar amino acids may be converted into polar amino acids (threonine, asparagine, glutamine, cysteine, tyrosine, aspartic acid, glutamic acid or histidine) or the parent amino acid may be changed to an alanine.

In embodiments, the method includes maintaining the temperature at about 55° C. In embodiments, the method includes maintaining the temperature at about 55° C. to about 80° C. In embodiments, the method includes maintaining the temperature at about 60° C. to about 70° C. In embodiments, the method includes maintaining the temperature at about 65° C. to about 75° C. In embodiments, the method includes maintaining the temperature at about 65° C. In embodiments, the method includes maintaining the temperature at about 60° C. In embodiments, the method includes maintaining the temperature at a pH of 8.0 to 11.0. In embodiments, the pH is 9.0 to 11.0. In embodiments, the pH is 9.5. In embodiments, the pH is 10.0. In embodiments, the pH is 8.0, 8.5, 9.0, 9.5, 10.0, 10.5, or 11.0. In embodiments, the pH is from 9.0 to 11.0, and the temperature is about 60° C. to about 70° C. In embodiments, the pH is from 8.5 to 9.5, and the temperature is about 58° C. to about 62° C.

In embodiments, the polymerases described herein have improved polymerase activity (i.e., improved relative to a control). Polymerase activity, in some instances, includes the measurable quantity k_{cat}, k_{cat}/Kₘ, or yields of incorporated nucleotides for a given time period. In embodiments, the polymerases described herein have increased extension activity (i.e., increased relative to a control). Increased extension activity variously refers to an increase in reaction kinetics (increased k_{cat}), increased K_{D}, decreased Kₘ, increased k_{cat}/Kₘ ratio, faster turnover rate, higher turnover number, or other metric that is beneficial to the use of the polypeptide for nucleic acid extension with nucleotides. The polypeptides described herein often incorporate at least 30% more nucleotides than the wild-type polymerase in total or in a given duration of time.

In embodiments, the polymerases described herein often incorporate at least 10%, 20%, 30%, 50%, 75%, 100%, 125%, 150%, 200%, 500%, more nucleotides than a control (e.g., the wild-type polymerase) for a fixed amount of time and same nucleotide concentration. In embodiments, the polymerases described herein incorporate nucleotides at least 1.5, 2, 2.5, 5, 10, 15, 20, 25, or at least 50 times faster than a control (e.g., the wild-type polymerase) for a fixed amount of time. Such measurements are often measured under conditions such as a set period of time, such as at least, at most, or exactly 1, 2, 3, 5, 8, 10, 15, 20, or more than 20 minutes. Such measurements are often measured under conditions such as a set nucleotide concentration, such as less than 10 μM, 10 μM, 20 μM, 50 μM, 100 μM, 200 μM, 300 μM, 500 μM, or more than 500 μM, or any concentration within the range identified herein.

In an aspect is provided a method of sequencing a circular polynucleotide. In embodiments, the method includes circularizing a linear nucleic acid molecule to form a circular polynucleotide. In embodiments, the circularizing includes intramolecular joining of the 5′ and 3′ ends of a linear nucleic acid molecule. In embodiments, the circularizing includes a ligation reaction. In embodiments, the two ends of the linear nucleic acid molecule are ligated directly together. In embodiments, the two ends of the linear nucleic acid molecule are ligated together with the aid of a bridging oligonucleotide (sometimes referred to as a splint oligonucleotide) that is complementary with the two ends of the linear nucleic acid molecule. Methods for forming circular DNA templates are known in the art, for example, linear polynucleotides are circularized in a non-template driven reaction with circularizing ligase, such as CircLigase™, CircLigase™ II, Taq DNA Ligase, HiFi Taq DNA Ligase, T4 DNA ligase, or Ampligase® DNA Ligase. In some embodiments, circularization is facilitated by denaturing double-stranded linear nucleic acids prior to circularization. Residual linear DNA molecules may be optionally digested. In some embodiments, circularization is facilitated by chemical ligation (e.g., click chemistry, e.g., a copper-catalyzed reaction of an alkyne (e.g., a 3′ alkyne) and an azide (e.g., a 5′ azide)). In embodiments, prior to circularization, the linear DNA fragments are A-tailed (e.g., A-tailed using Taq DNA polymerase).

In embodiments, circularization of the linear nucleic acid molecule is performed with CircLigase™ enzyme. In embodiments, circularization of the linear nucleic acid molecule is performed with a thermostable RNA ligase, or mutant thereof. In embodiments, circularization of the linear nucleic acid molecule is performed with an RNA ligase enzyme from bacteriophage TS2126, or mutant thereof. For example, the RNA ligase may be TS2126 RNA ligase, as described in U.S. Pat. Pub. 2005/0266439, which is incorporated herein by reference in its entirety.

In embodiments, circularizing includes ligating a first hairpin and a second hairpin adapter to a linear nucleic acid molecule, thereby forming a circular polynucleotide.

In embodiments, a hairpin adapter includes a single nucleic acid strand including a stem-loop structure. A hairpin adapter can be any suitable length. In some embodiments, a hairpin adapter is at least 40, at least 50, or at least 100 nucleotides in length. In some embodiments, a hairpin adapter has a length in a range of 45 to 500 nucleotides, 75-500 nucleotides, 45 to 250 nucleotides, 60 to 250 nucleotides or 45 to 150 nucleotides. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, the second adapter includes a sample barcode sequence, a molecular identifier sequence, or both a sample barcode sequence and a molecular identifier sequence. In some embodiments, the second adapter includes a sample barcode sequence.

In some embodiments, a duplex region or stem portion of a hairpin adapter includes an end that is configured for ligation to an end of double stranded nucleic acid (e.g., a nucleic acid fragment, e.g., a library insert). In embodiments, an end of a duplex region or stem portion of a hairpin adapter includes a 5′-overhang or a 3′-overhang that is complementary to a 3′-overhang or a 5′-overhang of one end of a double stranded nucleic acid. In some embodiments, an end of a duplex region or stem portion of a hairpin adapter includes a blunt end that can be ligated to a blunt end of a double stranded nucleic acid. In certain embodiment, an end of a duplex region or stem portion of a hairpin adapter includes a 5′-end that is phosphorylated. In some embodiments, a stem portion of a hairpin adapter is at least 15, at least 25, or at least 40 nucleotides in length. In some embodiments, a stem portion of a hairpin adapter has a length in a range of 15 to 500 nucleotides, 15-250 nucleotides, 15 to 200 nucleotides, 15 to 150 nucleotides, 20 to 100 nucleotides or 20 to 50 nucleotides.

In some embodiments, the loop of a hairpin adapter includes one or more of a primer binding site, a capture nucleic acid binding site (e.g., a nucleic acid sequence complementary to a capture nucleic acid), a UMI, a sample barcode, a sequencing adapter, a label, the like or combinations thereof. In certain embodiments, a loop of a hairpin adapter includes a primer binding site. In certain embodiments, a loop of a hairpin adapter includes a primer binding site and a UMI. In certain embodiments, a loop of a hairpin adapter includes a binding motif.

In some embodiments, the loop of a hairpin adapter has a predicted, calculated, mean, average or absolute melting temperature (Tm) that is greater than 50° C., greater than 55° C., greater than 60° C., greater than 65° C., greater than 70° C. or greater than 75° C. In some embodiments, a loop of a hairpin adapter has a predicted, estimated, calculated, mean, average or absolute melting temperature (Tₘ) that is in a range of 50-100° C., 55-100° C., 60-100° C., 65-100° C., 70-100° C., 55-95° C., 65-95° C., 70-95° C., 55-90° C., 65-90° C., 70-90° C., or 60-85° C. In embodiments, the Tm of the loop is about 65° C. In embodiments, the Tm of the loop is about 75° C. In embodiments, the Tm of the loop is about 85° C. The Tm of a loop of a hairpin adapter can be changed (e.g., increased) to a desired Tm using a suitable method, for example by changing (e.g., increasing GC content), changing (e.g., increasing) length and/or by the inclusion of modified nucleotides, nucleotide analogues and/or modified nucleotides bonds, non-limiting examples of which include locked nucleic acids (LNAs, e.g., bicyclic nucleic acids), bridged nucleic acids (BNAs, e.g., constrained nucleic acids), C5-modified pyrimidine bases (for example, 5-methyl-dC, propynyl pyrimidines, among others) and alternate backbone chemistries, for example peptide nucleic acids (PNAs), morpholinos, the like or combinations thereof. Accordingly, in some embodiments, a loop of a hairpin adapter includes one or more modified nucleotides, nucleotide analogues and/or modified nucleotides bonds.

In some embodiments, the loop of a hairpin adapter independently includes a GC content of greater than 40%, greater than 50%, greater than 55%, greater than 60% greater than 65% or greater than 70%. In certain embodiments, a loop of a hairpin adapter independently includes a GC content in a range of 40-100%, 50-100%, 60-100% or 70-100%. In embodiments, the loop has a GC content of about or more than about 40%. In embodiments, the loop has a GC content of about or more than about 50%. In embodiments, the loop has a GC content of about or more than about 60%. Non-base modifiers can also be incorporated into a loop of a hairpin adapter to increase Tₘ, non-limiting examples of which include a minor grove binder (MGB), spermine, G-clamp, a Uaq anthraquinone cap, the like or combinations thereof. A loop of a hairpin adapter can be any suitable length. In some embodiments, a loop of a hairpin adapter is at least 15, at least 25, or at least 40 nucleotides in length. In some embodiments, a hairpin adapter has a length in a range of 15 to 500 nucleotides, 15-250 nucleotides, 20 to 200 nucleotides, 30 to 150 nucleotides or 50 to 100 nucleotides.

In certain embodiments, a duplex region or stem region of a hairpin adapter includes a predicted, estimated, calculated, mean, average or absolute Tm in a range of 30-70° C., 35-65° C., 35-60° C., 40-65° C., 40-60° C., 35-55° C., 40-55° C., 45-50° C. or 40-50° C. In embodiments, the Tm of the stem region is about or more than about 35° C. In embodiments, the Tm of the stem region is about or more than about 40° C. In embodiments, the Tm of the stem region is about or more than about 45° C. In embodiments, the Tm of the stem region is about or more than about 50° C.

In one embodiment, an enzyme is used to ligate the two ends of the linear nucleic acid molecule. For example, linear polynucleotides are circularized in a non-template driven reaction with a circularizing ligase, such as CircLigase™ enzyme, Taq DNA Ligase, HiFi Taq DNA Ligase, T4 DNA ligase, PBCV-1 DNA Ligase (also known as SplintR ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, the ligase enzyme includes a T4 DNA ligase, T4 RNA ligase 1, T4 RNA ligase 2, T3 DNA ligase or T7 DNA ligase. In embodiments, the enzymatic ligation is performed by a mixture of ligases. In embodiments, the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase (e.g., 5′AppDNA/RNA ligase), an ATP dependent DNA ligase, an RNA-dependent DNA ligase (e.g., SplintR ligase), and combinations thereof. In embodiments, the two ends of the template polynucleotide are ligated together with the aid of a splint primer that is complementary with the two ends of the template polynucleotide. For example, a T4 DNA ligase reaction may be carried out by combining a linear polynucleotide, ligation buffer, ATP, T4 DNA ligase, water, and incubating the mixture at between about 20° C. to about 45° C., for between about 5 minutes to about 30 minutes. In some embodiments, the T4 ligation reaction is incubated at 37° C. for 30 minutes. In some embodiments, the T4 ligation reaction is incubated at 45° C. for 30 minutes. In embodiments, the ligase reaction is stopped by adding Tris buffer with high EDTA and incubating for 1 minute.

In embodiments, a linear nucleic acid molecule may undergo intramolecular circularization (via ligation or annealing) without joining to a circularization adapter (e.g., self-circularization). Circularization (without a circularization adaptor) can be achieved with a ligase at about 4°-35° C. In embodiments, a linear nucleic acid molecule interest can be joined to a loxP adapter and circularization can be mediated by a Cre recombinase enzyme reaction at about 4°-35° C., see for example U.S. Pat. No. 6,465,254, which is incorporated herein by reference.

In embodiments, the circular polynucleotide that is about 100 to about 1000 nucleotides in length, about 100 to about 300 nucleotides in length, about 300 to about 500 nucleotides in length, or about 500 to about 1000 nucleotides in length. In embodiments, the circular polynucleotide is about 300 to about 600 nucleotides in length. In embodiments, the circular polynucleotide is about 100-1000 nucleotides, about 150-950 nucleotides, about 200-900 nucleotides, about 250-850 nucleotides, about 300-800 nucleotides, about 350-750 nucleotides, about 400-700 nucleotides, or about 450-650 nucleotides in length. In embodiments, the circular polynucleotide molecule is about 100-1000 nucleotides in length. In embodiments, the circular polynucleotide molecule is about 100-300 nucleotides in length. In embodiments, the circular polynucleotide molecule is about 300-500 nucleotides in length. In embodiments, the circular polynucleotide molecule is about 500-1000 nucleotides in length. In embodiments, the circular polynucleotide molecule is about 100 nucleotides. In embodiments, the circular polynucleotide molecule is about 300 nucleotides. In embodiments, the circular polynucleotide molecule is about 500 nucleotides. In embodiments, the circular polynucleotide molecule is about 1000 nucleotides. Circular polynucleotides may be conveniently isolated by a conventional purification column, digestion of non-circular DNA by one or more appropriate exonucleases, or both.

In embodiments, the sequencing includes sequencing by synthesis, sequencing-by-binding, sequencing by hybridization, sequencing by ligation, or pyrosequencing. A variety of sequencing methodologies can be used such as sequencing-by synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting of steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by a sequencing-by-synthesis (SBS) process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 7,541,444, 7,057,026, and 10,738,072. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Sequencing can be carried out using any suitable sequencing-by-synthesis (SBS) technique, wherein modified nucleotides are added successively to a free 3′ hydroxyl group, typically initially provided by a sequencing primer, resulting in synthesis of a polynucleotide chain in the 5′ to 3′ direction. In embodiments, sequencing includes detecting a sequence of signals. In embodiments, sequencing includes extension of a sequencing primer with labeled nucleotides. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

In embodiments, generating a first sequencing read or a second sequencing read includes sequencing-by-binding (see, e.g., U.S. Pat. Pubs. US2017/0022553 and US2019/0048404, each of which is incorporated herein by reference in its entirety). As used herein, “sequencing-by-binding” refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, the sequencing includes a plurality of sequencing cycles. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove label(s) from each incorporated base. Reagents, enzymes and other substances can be removed between steps by washing. Cycles may include repeating these steps, and the sequence of each cluster is read over the multiple repetitions. In embodiments, the sequencing yields reads of greater than 25 bp read length. In embodiments, the sequencing yields reads of greater than 50 bp read length. In embodiments, the sequencing yields reads of greater than 75 bp read length. In embodiments, the sequencing yields reads of greater than 100 bp read length. In embodiments, the sequencing yields reads of greater than 150 bp read length. In embodiments, generating a sequencing read includes determining the identity of the nucleotides in the template polynucleotide. In embodiments, a sequencing read, e.g., a first sequencing read or a second sequencing read, includes determining the identity of a portion (e.g., 1, 2, 5, 10, 20, 50 nucleotides) of the total template polynucleotide. In embodiments the first sequencing read determines the identity of 5-10 nucleotides and the second sequencing read determines the identity of more than 5-10 nucleotides (e.g., 11 to 200 nucleotides). In embodiments the first sequencing read determines the identity of more than 5-10 nucleotides (e.g., 11 to 200 nucleotides) and the second sequencing read determines the identity of 5-10 nucleotides.

In embodiments, the sequencing method relies on the use of modified nucleotides that can act as reversible terminators. Once the modified nucleotide has been incorporated into the growing polynucleotide chain complementary to the region of the template being sequenced there is no free 3′-OH group available to direct further sequence extension and therefore the polymerase cannot add further nucleotides. Once the identity of the base incorporated into the growing chain has been determined, the 3′ reversible terminator may be removed to allow addition of the next successive nucleotide. These such reactions can be done in a single experiment if each of the modified nucleotides has attached a different label, known to correspond to the particular base, to facilitate discrimination between the bases added at each incorporation step. Alternatively, a separate reaction may be carried out containing each of the modified nucleotides separately.

The modified nucleotides may carry a label (e.g., a fluorescent label) to facilitate their detection. Each nucleotide type may carry a different fluorescent label. However, the detectable label need not be a fluorescent label. Any label can be used which allows the detection of an incorporated nucleotide. One method for detecting fluorescently labeled nucleotides includes using laser light of a wavelength specific for the labeled nucleotides, or the use of other suitable sources of illumination. The fluorescence from the label on the nucleotide may be detected (e.g., by a CCD camera or other suitable detection means).

In embodiments, the methods of sequencing a nucleic acid include extending a complementary polynucleotide (e.g., a primer) that is hybridized to the nucleic acid by incorporating a first nucleotide (e.g., a modified, labeled nucleotide). In embodiments, the method includes a buffer exchange or wash step. In embodiments, the methods of sequencing a nucleic acid include a sequencing solution. The sequencing solution includes (a) an adenine nucleotide, or analog thereof; (b) (i) a thymine nucleotide, or analog thereof, or (ii) a uracil nucleotide, or analog thereof; (c) a cytosine nucleotide, or analog thereof; and (d) a guanine nucleotide, or analog thereof.

In embodiments, the sequencing includes extending a sequencing primer by incorporating a labeled nucleotide, or labeled nucleotide analogue, and detecting the label to generate a signal for each incorporated nucleotide or nucleotide analogue.

In embodiments, the method includes amplifying the template polynucleotide in a cell. In embodiments, the method includes amplifying the template polynucleotide in a tissue. In embodiments, the method includes amplifying the template polynucleotide one a solid support (e.g., a multiwell container or a flowcell). In embodiments, the amplification primer is immobilized on a solid support.

The methods and kits of the present disclosure may be applied, mutatis mutandis, to the sequencing of RNA, or to determining the identity of a ribonucleotide.

### EXAMPLES

### Example 1. Focused Mutational Analysis

An aim of the general experimental plan was to produce a robust, optimized polymerase for nucleic acid sequencing methods. DNA polymerases of the Pyrococcus genus share similar anerobic features as other thermophilic genera (e.g., Archaeoglobus, Thermoautotrophican, Methanococcus), however, Pyrococcus species thrive in higher temperatures, ca 100° C., and tolerate extreme pressures. For example, the area around undersea hot vents, where P. abyssi has been found, there is no sunlight, the temperature is around 98° C.-100° C. and the pressure is about 200 atm. These Pyrococcus polymerases possess inherent properties that are beneficial for sequencing applications.

Directed evolution of enzymes is a process that mimics natural selection in vitro. Compartmentalized self-replication (CSR) is a method of directed evolution where a library containing mutated variants of the enzyme of interest goes through rounds of selective pressure, and over time, the most active or best performing variants are enriched in the library, compared to less active variants, as described in Abil, Z., & Ellington, A. D. (2018). Current Protocols in Chemical Biology, 10, 1-17. During CSR, the enzyme variants and its own encoding genes are compartmentalized in oil emulsions, together with dNTPs and primers. During the emulsion PCR, each enzyme that can surpass the selective pressure is able to replicate its own encoding gene and pass to the next round of selection. Over time, the best performers are enriched in the library.

DNA polymerases carry out crucial functions in many DNA metabolic processes, and due to their ability to catalyze the replication of DNA by incorporating nucleotides into the 3′ end of a primer annealed to a template, DNA polymerases are frequently used in genomic research (e.g., next-generation sequencing, or NGS, technologies). The human genome encodes at least 14 DNA-dependent DNA polymerases, each serving a particular function. The general classification includes five different classes according to their function: DNA polymerase (Pol α) catalyzes DNA replication at Okazaki fragments on the lagging strand; Pol β participates in base-excision repair; Pol γ is involved in mitochondrial DNA synthetic processes; Pol δ participates in lagging-strand synthesis; and Pol δ catalyzes the synthesis of the leading strand of chromosomal DNA.

In the context of nucleic acid sequencing, the use of nucleotides bearing a 3′ reversible terminator allows successive nucleotides to be incorporated into a polynucleotide chain in a controlled manner. The DNA template for a sequencing reaction will typically comprise a double-stranded region having a free 3′ hydroxyl group which serves as a primer or initiation point for the addition of further nucleotides in the sequencing reaction. The region of the DNA template to be sequenced will overhang this free 3′ hydroxyl group on the complementary strand. The primer bearing the free 3′ hydroxyl group may be added as a separate component (e.g., a short oligonucleotide) which hybridizes to a region of the template to be sequenced. Following the addition of a single nucleotide to the DNA template, the presence of the 3′ reversible terminator prevents incorporation of a further nucleotide into the polynucleotide chain. While the addition of subsequent nucleotides is prevented, the identity of the incorporated is detected (e.g., exciting a unique detectable label that is linked to the incorporated nucleotide). The reversible terminator is then removed, leaving a free 3′ hydroxyl group for addition of the next nucleotide. The sequencing cycle can then continue with the incorporation of the next blocked, labelled nucleotide. Sequencing by synthesis of nucleic acids ideally requires the controlled (i.e., one at a time), yet rapid, incorporation of the correct complementary nucleotide opposite the oligonucleotide being sequenced. This allows for accurate sequencing by adding nucleotides in multiple cycles as each nucleotide residue is sequenced one at a time, thus preventing an uncontrolled series of incorporations occurring.

As described herein wild-type Pyrococcus enzymes (e.g., P. horikoshii and P. abyssi) have difficulty incorporating modified nucleotides (e.g., nucleotides including a reversible terminator and/or a cleavable linked base). Relative to a non-modified nucleotide, an incoming modified nucleotide bearing a 3′ reversible terminator increases the activation energy required to orient the phosphate for phosphoryl transfer. To efficiently incorporate modified nucleotides, the DNA polymerase active site needs to be engineered to accommodate a variety of nucleotide structural variants. DNA polymerases evolved mechanisms to ensure selection of the correct nucleotide in order to maintain the integrity and fidelity of the nucleic acid sequence. One such mechanism is the highly conserved region in family B DNA polymerases active site, which includes the amino acids LYP at positions 408-410 of 9°N polymerases. The modifications at amino acid positions D141 and E143 (relative to wild-type) are known to affect exonuclease activity (designated exo-) (see, for example, U.S. Pat. No. 5,756,334 and Southworth et al, 1996 Proc. Natl Acad. Sci USA 93:5281). This 3′-5′ exonuclease activity is absent in some DNA polymerases (e.g., Taq DNA). It is typically beneficial to remove this exonuclease proof-reading activity when using modified nucleotides to prevent the exonuclease removing the unnatural nucleotide after incorporation.

Additional mutations to wild type DNA polymerase enzymes are useful for DNA sequencing applications involving 3′ modified nucleotides. Such changes have previously been made for the Vent and Deep Vent DNA polymerases. As described in WO 2005/024010, modifications to the so-called motif A region, amino acid positions 408-410 of 9°N polymerases, exhibit improved incorporation of nucleotide analogues bearing substituents at the 3′ position of the sugar. Of note, amino acids at positions 408, 409, 410 in a 9°N polymerase are functionally equivalent to amino acids at positions 409, 410, and 411 in wild type P. abyssi and P. horikoshii. This trio of amino acids are in close proximity to the nucleotide that is being incorporated and is strictly conserved across the different types of Family B polymerases; see for example US 2017/0298327 A1; Gueguen, Y., et al (2001), European Journal of Biochemistry, 268:5961-5969; and Bergen, K., et al. (2013), ChemBioChem, 14:1058-1062, which are incorporated herein in its entirety for all purposes. Because these three amino acids are in close proximity to the nucleotide being incorporated, a change in the sequence or structure of this motif alters the incorporation kinetics. The amino acids at positions 408, 409, 410 in a 9°N polymerase and Vent™ polymerase are positionally equivalent (i.e., the amino acids at positions 408, 409, 410 in a 9°N polymerase correspond) to amino acids 409, 410, and 411 in wild type P. abyssi, and play an important role in incorporating a modified nucleotide into a primer. Significant strides in DNA sequencing have been achieved through engineered DNA polymerases. U.S. Pat. Nos. 11,136,565, 11,845,932, and 11,884,943, each of which are incorporated herein by reference, provides mutant Pyrococcus polymerases, enhancing their ability to incorporate reversible terminator nucleotides.

Structural analyses of DNA polymerases portray the enzyme as analogous to a human right hand, with three domains: a ‘fingers’ domain that interacts with the incoming dNTP and paired template base, and that closes at each nucleotide addition step; a ‘palm’ domain that catalyzes the phosphoryl-transfer reaction; and a ‘thumb’ domain that interacts with duplex DNA. The finger and palm subdomains of DNA polymerases (e.g., amino acids positions 448-603 of SEQ ID NO:1) are in close proximity to the nucleotide incorporation region.

For brevity, amino acid mutation nomenclature is used throughout this application. One having skill in the art would understand the amino acid mutation nomenclature, such that D141A refers to aspartic acid (single letter code is D), at position 141, is replaced with alanine (single letter code A). Likewise, it is understood that when an amino acid mutation nomenclature is used and the terminal amino acid code is missing, e.g., P411, it is understood that no mutation was made relative to the wild type. Additionally, for amino acid positions that are frequently mutated herein the wild type amino acid may be recited to emphasize that it is not mutated, for example P411P.

The initial library consisted of copies of a mutant Pyrococcus horikoshii polymerase, wherein the point mutations have been described previously (e.g., U.S. Pat. Nos. 11,136,565, 11,034,942, and U.S. Pat. No. 11,88,943), in a pET21b+ vector. Seven rounds of Compartmentalized Self Replication were carried out as described in Abil & Ellington (Abil, Z., Ellington, A. D. (2018). Current protocols in chemical biology, 10(1), 1-17). The initial plasmid library was transformed into T7 express electrocompetent E. coli cells made in-house. The transformed cells were cultured overnight at 37° C. in 50 ml centrifuge tubes containing 10 ml of LB media containing antibiotic. On the next day, 100-ml Erlenmeyer flasks containing 20 ml of LB+antibiotic were inoculated with the starter culture and grown at 37 C. The absorbance of the culture was measured using a Quickdrop, and when the OD600 was ˜0.7, protein expression was induced by adding 0.5 mM IPTG. At this time, the temperature was adjusted to 18° C., and the expression was carried overnight.

A water-in-oil emulsion using known techniques, for example as described in Povilaitis. T. et al. (2016). Protein Engineering, Design and Selection, Volume 29, Issue 12, 28 Dec. 2016, Pages 617-628. The oil/surfactant mixture consisted of mineral oil, while the solution phase consisted of the Pyrococcus enzyme DNA polymerase in a buffer pH 8.0 containing 0.5 μM of each CSR primer, 250 μM of each dNTP, and 1×10⁸ E. coli cells containing the plasmid library and the expressed proteins. The oil and liquid phases were combined in 1.5 ml Eppendorf tubes containing a magnetic mini stir bar, and the emulsion was formed using a Tissuelyzer for 10 min. The emulsion PCR program was designed according to the specific CSR primers used for each round.

The emulsions were washed with diethyl ether and ethyl acetate, followed by a DNA clean-up step using the Monarch® PCR clean and concentrate kit (New England Biolabs™). The extracted liquid phase was treated with DPN1 to remove the parental plasmids from the reaction, leaving only the products of amplification of that specific selection round. A new PCR reaction is performed to further amplify the products from the Emulsion PCR. Here we utilize the Q5® High-Fidelity Master mix (NEB) containing 0.5 μM of each Recovery primer. Recovery primers and thermocycling programs varied based on the CSR primers used. The product of Recovery PCR is purified from agarose gels using the Zymo gel extraction kit (Zymo Research), followed by an extra purification step using the Monarch® PCR cleanup kit. A new PCR reaction is performed in addition to the Recovery PCR to remove the non-gene-specific “handles” present in the CSR primers and Recovery primers. The process is identical to that of the Recovery PCR, except for the primers and thermocycling programs used.

Cloning of the enriched amplicons into a vector was done via [i] restriction digestion and ligation, or [ii] multi-fragment Gibson assembly. The enriched plasmid libraries were cloned into E. coli cells as described earlier, and a new round of selection took place.

Restriction digestion and ligation: This method was used on rounds 1 through 5. The products from Re-Amp PCR were digested with EcoRI-HF and XhoI in Cutsmart Buffer. The reaction was incubated for 1 h at 37° C., and the product was purified. In parallel, a pET21b+ vector fragment was prepared by cutting the pET21b+ with the same restriction enzymes EcoRI-HF and XhoI. The library was cloned into the vector fragment using T4 DNA Ligase at room temperature for 20 minutes. Multi-fragment Gibson Assembly: This method was used on rounds 6 and 7. The product of Re-Amp PCR was amplified by 3 different primer pairs in 3 separate thermocycling reactions. Each primer pair amplified a fragment of the gene, with a approximately 20 bp overlap. Amplicons were purified from agarose gels, followed by an additional purification. In parallel, a pD454-SR (ATUM) vector fragment was prepared by amplifying the commercial pD454 with primers containing an approximately 20 bp overlap to the outermost gene fragments. The resulting gene fragments were cloned into the pD454 vector fragment via Gibson Assembly, containing 100 ng of vector fragment and 0.05 pmol of each gene fragment. Reactions were incubated at 50 C for 1 h and purified.

Selective pressures: The ability to amplify DNA in the emulsion PCR is the first selective pressure. The starting DNA polymerase gene had in the exonuclease domain two mutations (D141A and E143A), that removed the exonuclease activity of the enzyme. Since the first DNA polymerase to be produced in CSR had low fidelity, it introduced mutations when amplifying its own gene. The emulsion PCR on its own acted as the main selective pressure to develop a higher accuracy polymerase. The “self-generated mutant library” of new polymerases, went into the next round of selection. From there on, low accuracy variants fall off from the selection due to poor ability to replicate its own gene, while high accuracy variants succeed in amplifying their gene after each round, leading to a final library enriched with high accuracy variants. That is, only the enzymes capable of replicating its own encoding gene, would be enriched in the library.

To promote high fidelity, the selective pressures included modulating the annealing and extension temperature for 27 PCR cycles. As described in Table 1, the annealing and extension temperatures of emulsion PCR were gradually lowered, and the duration of extension was also reduced to select for polymerases with fast incorporation and strong amplification at temperatures greater than 60° C. in addition to high-accuracy incorporation. For the final two rounds, Round 6 and 7, the extension time was reduced from 6 minutes to 4.5 minutes.

TABLE 1
A summary of the selective pressures applied per round of CSR.

| | Round 1 | | Round 2 | | Round 3 | | Round 4 | | Round 5 | | Round 6 | | Round 7 | |
|---|---|---|---|---|---|---|---|---|---|---|---|---|---|---|
| Annealing, | 72° | C. | 72° | C. | 68° | C. | 65° | C. | 61° | C. | 61° | C. | 61° | C. |
| Extension, | | | | | | | | | | | | | | |
| and Final | | | | | | | | | | | | | | |
| Extension | | | | | | | | | | | | | | |
| temperature | | | | | | | | | | | | | | |
| Extension | 6 | min | 6 | min | 6 | min | 6 | min | 6 | min | 4.5 | min | 4.5 | min |
| duration | | | | | | | | | | | | | | |

Polymerases were isolated, purified, and tested in fidelity and incorporation assays further described herein.

### Example 2. Measuring Enzyme Fidelity and Incorporation

The fidelity of a DNA polymerase is the result of accurate replication of a desired template. Specifically, this involves multiple steps, including the ability to read a template strand, select the appropriate nucleoside triphosphate and insert the correct nucleotide at the 3′ primer terminus, such that Watson-Crick base pairing is maintained. In addition to effective discrimination of correct versus incorrect nucleotide incorporation, some DNA polymerases possess a 3′->5′ exonuclease activity. This activity, known as “proofreading”, is used to excise incorrectly incorporated mononucleotides that are then replaced with the correct nucleotide. In embodiments of the invention described herein, the exonuclease activity has been removed, therefore it is important to have a high-fidelity enzyme.

High-fidelity DNA polymerases have safeguards to protect against both making and propagating mistakes while copying DNA. Such mutated polymerases have a significant binding preference for the correct versus the incorrect nucleotide during polymerization. Fidelity of the polymerase may be quantified using any suitable method known in the art. For example, to quantify the fidelity herein, the method includes performing a single nucleotide extension where the next base to be incorporated is known (e.g., A) in the presence of excess incorrect nucleotide (e.g., G). For example, the enzyme, template, primer composition is mixed with 5 mM dATP and 500 mM dGTP (the most likely misincorporation), to probe nucleotide incorporation with 100-fold excess of the wrong nucleotide. The reported fidelity percentage is the signal (relative fluorescence units) from the correct base normalized by the total signal. For example, when measuring fidelity on a template that expects an “A” to be incorporated, the fidelity % would be the ratio of (“A” signal)/(“A”+ “T”+ “C”+ “G” signals), multiplied by 100. Therefore, a higher fidelity score corresponds to a lower rate of misincorporation (i.e., incorporating the incorrect nucleotide).

The rate of incorporation of a fluorescent nucleotide reversible terminator (NRT) was measured using primer/templates attached to avidin-coated magnetic beads (MyOne C1, ThermoFisher). The 5′-biotinylated 160 primer is annealed to the appropriate 160-X template and bound to the beads along with a tethering oligo 5′-Biotin-CG(TAGCCG)₆TAGC-3ddC (tether B). The beads are then attached to the surface of 384-well streptavidin-coated plates (Greiner Bio-one) to which tether A (5′-Biotin-GC (TACGGC)₆TACG-3ddC) has previously been bound. Reactions are initiated in a house-developed buffer by the addition of 100 nM nucleotides (or 300 nM nucleotides for Challenge template sequences, unless otherwise indicated) and 133 nM DNA polymerase at a temperature of 61° C. The reaction is stopped by flooding duplicate wells with room temperature wash buffer after incubation for 15 seconds and additional wells after 10 minutes. Blanks were also made without incubation. The wells were imaged under a fluorescence microscope, and the images analyzed using software that identifies fluorescent beads and calculates their average brightness. The blank was subtracted from the time points and the values at 15 seconds and 10 minutes used to calculate the half-time of incorporation assuming first-order kinetics with completion in under 10 minutes.

TABLE 2
General Template Sequences

| | |
|---|---|
| 160-1 | 5′ - |
| (SEQ ID NO: 2) | GACTCACATGAATCAGTGCAGCATCAGATGTATGACCGAAGCGGACGAAGG |
| | TGCGTGGA-3ddC |
| 160-2 | 5′ - |
| (SEQ ID NO: 3) | GTGGTTCATCGCGTCCGATATCAAACTTCGTCAAGTCGAAGCGGACGAAGG |
| | TGCGTGGA-3ddC |
| 160-3 | 5′ - |
| (SEQ ID NO: 4) | TACTAGGTTGTACGATCCCTGCACTTCAGCTAAGCACGAAGCGGACGAAGG |
| | TGCGTGGA-3ddC |
| 160-4 | 5′ - |
| (SEQ ID NO: 5) | AGCTACCAATATTTAGTTTCCGAGTCTCAGCTCATGCGAAGCGGACGAAGG |
| | TGCGTGGA-3ddC |
| 160 Primer | 5′ -Biotin- |
| (SEQ ID NO: 6) | AAAAAAAAAAAAGTCCACGCACCTTCGTCCGCTTCG |

The underlined nucleotide in Table 1 is the first one nucleotide downstream from the 160 primer.

Through ongoing SBS experiments, data shows that certain nucleic acid sequences in the template that precede the nucleotide about to be incorporated can temporarily stall or slow down incorporation of the next nucleotide. Generally, they are GC-rich sequences; for example, some difficult sequences in the template that precede nucleotide to be incorporated may be described in Table 2.

TABLE 3
Difficult sequences

| Nucleotide | Difficult sequences in the template |
|---|---|
| to be | that precede the complementary |
| incorporated | nucleotide to be incorporated |
| T | 5′-CCGCC (SEQ ID NO: 7) |
| G | 5′-GCGCT (SEQ ID NO: 8) |
| A | 5′-CCGCG (SEQ ID NO: 9) |
| C | 5-ACGCC (SEQ ID NO: 10) |

Therefore, a set of templates, dubbed ‘challenge-templates,’ were devised to assist in identifying polymerase mutants capable of rapid nucleotide incorporation. An example of the challenge template sequences are listed in Table 4, and the assay conditions are the same as the conditions used for the General Template sequences provided in Table 1. To note, the underlined sequences in the challenge-templates correspond to the difficult sequences identified in Table 2, while the bold nucleotide refers to the nucleotide complement to be incorporated.

TABLE 4
Challenge Template Sequences

| | |
|---|---|
| 260-1 | 5′ - |
| (SEQ ID NO: 11) | CCAACTTGATATTAATAACACTATAGACCACCGCCCGAAGCGGACGAAGGT |
| | GCGTGGA/3ddC/ |
| 260-2 | 5′ - |
| (SEQ ID NO: 12) | ATGATTAAACTCCTAAGCAGAAAACCTACCGCGCTCGAAGCGGACGAAGGT |
| | GCGTGGA/3ddC/ |
| 260-3 | 5′ - |
| (SEQ ID NO: 13) | TCTTTAATAACCTGATTCAGCGAAACCAATCCGCGCGAAGCGGACGAAGGT |
| | GCGTGGA/3ddC/ |
| 260-4 | 5′ - |
| (SEQ ID NO: 14) | CGGTTATCGCTGGCGACTCCTTCGAGATGGACGCCCGAAGCGGACGAAGGT |
| | GCGTGGA/3ddC/ |
| 260-1 Primer | 5′ - |
| (SEQ ID NO: 15) | Bio/AAAAAAAAAAAAGTCCACGCACCTTCGTCCGCTTCGGGCGG |
| 260-2 Primer | 5′ - |
| (SEQ ID NO: 16) | Bio/AAAAAAAAAAAAGTCCACGCACCTTCGTCCGCTTCGAGCGC |
| 260-3 Primer | 5′ - |
| (SEQ ID NO: 17) | Bio/AAAAAAAAAAAAGTCCACGCACCTTCGTCCGCTTCGCGCGG |
| 260-4 Primer | 52- |
| (SEQ ID NO: 18) | Bio/AAAAAAAAAAAAGTCCACGCACCTTCGTCCGCTTCGGGCGT |

The sequencing data from each mutant was analyzed to identify mutations in the nucleotide level, which were then translated to amino acids. The amino acid mutations calculated frequency of each mutation per round was obtained. The CSR Library was narrowed over the rounds of selection and shows many enriched mutations that are involved in strand-displacement. After each round of selection, the sequence of the enzyme was obtained to elucidate which mutations are responsible for the strand-displacement activity. Table 5 provides an overview of some of the mutations responsible for increased fidelity. Using the CSR techniques, novel mutations in a DNA polymerase were found. For example, the mutations identified in the top 6 mutant enzymes are identified in Table 5.

TABLE 5
Summary of point mutations identified in high-fidelity mutants;
the point mutations are relative to SEQ ID NO: 1.

| | Percentage of top | |
|---|---|---|
| Point | performing mutants | |
| mutation | containing this mutation | Internal Ref |
| E306G | 100% | BK-1, BK2-, BK-3, |
| | | BK-4, BK-5, BK-6 |
| V341L | 83% | BK-1, BK2-, BK-3, |
| | | BK-4, BK-5 |
| Y494F | 83% | BK-1, BK2-, BK-3, |
| | | BK-4, BK-5 |
| E581G | 83% | BK-1, BK2-, BK-3, |
| | | BK-4, BK-6 |
| F588L | 83% | BK-1, BK2-, BK-3, |
| | | BK-4, BK-6 |
| E280K | 67% | BK-1, BK2-, BK-4, |
| | | BK-5 |
| M241I | 50% | BK-1, BK-4, BK-6 |
| N236D | 33% | BK-1, BK-4 |

Though one parameter, the average half time of nucleotide incorporation is measured over all four nucleotides (A, T, C, and G), and serves as a useful indicator of the enzyme kinetics. Described in Table 6 is the average halftime, tv, averaged over each of the four incorporated modified nucleotides (i.e., A, T, C, and G) for halftime measurements using the General templates (i.e., the sequences described in Table 2) and the Challenge templates (i.e., the sequences described in Table 3). The mutants characterized in Table 6 all show an improvement in fidelity relative to a control polymerase. Some of the polymerases show an improved rate of incorporation (e.g., BK-1, and BK-4) and an increase in fidelity.

TABLE 6
Summary of fidelity and kinetics for the mutant
enzymes relative to control (e.g., SEQ ID NO: 1).

| | Average Incorporation | | |
|---|---|---|---|
| | Time in Challenge | | |
| | Templates +/− 1.0 | Average Fidelity in | Average Fidelity in |
| Internal Ref. | (reported in seconds) | General Template | Challenge Templates |
| BK-1 | 11.3 | 85.6% | 86.7% |
| BK-2 | 14.4 | 84.4% | 67.5% |
| BK-3 | 16.7 | 88.4% | 70.3% |
| BK-4 | 7.5 | 88.2% | 75.4% |
| BK-5 | 29.4 | | |
| BK-6 | 25.5 | 84.9% | 68.3% |
| Control | 12.0 | 73.9% | 65.7% |

BK-1 includes the point mutations (relative to SEQ ID NO:1): S46G; P104Q; K134R; N236D; S237N; M241I; I256F; F258L; L260F; F261L; E280K; I282F; E288D; E300G; E306G; V341L; T416A; V438L; LA79P; Y494F; E581G; and F588L.
BK-2 includes the point mutations (relative to SEQ ID NO:1): L85M; I206F; K221E; G233D; E280K; E306G; V341L; S348R; Y389H; 1415V; Y494F; Y498C; E581G; F588L; N636D; F749V; and W758R.
BK-3 includes the point mutations (relative to SEQ ID NO:1): I16V; V28A; Y30C; R101C; L145S; F152S; F214L; L219W; A249S; A277T; E280Q; E306G; V341L; Y389H; S452G; K466E; K469E; D473G; Y494F; E581G; F588L; L640A; K650R; N653T; and K773E.
BK-4 includes the point mutations (relative to SEQ ID NO:1): R35H; A117E; I206T; N236D; M241I; E280K; E306G; K310M; P340T; V341L; K469R; Y494F; E581G; F588L; T648A; I745T; F749S; K752N; and Q759R.
BK-5 includes the point mutations (relative to SEQ ID NO:1): M159T; K169E; 1176N; N210S; G233D; H257L; 1264V; E280K; E306G; E321G; F327L; L333S; V341L; G410V; K465R; K469R; K477E; K478E; R483L; Y494F; Y500C; W554R; Y580C; F589L; 1598V; T606A; E649G; K650R; T652A; R686L; 1700V; Y733H; V742A; F749S; K752N; Q759R; K773R; and K774E.
BK-6 includes the point mutations (relative to SEQ ID NO:1): 116V; E22D; K70R; K118R; K124R; L145S; 1171F; K175R; K199E; M241I; Q242R; L248P; P271S; E306G; E378V; K508R; E581G; F588L; K642E; 1671T; I745F; A748S; and W758R.

### Example 3: Cysteine-modified Pyrococcus Polymerases

Modified nucleotides that contain a unique cleavably-linked fluorophore and a reversible-terminating moiety capping the 3′-OH group, for example, those described in U.S. 2017/0130051, WO 2017/058953, WO 2019/164977, and U.S. Pat. No. 10,738,072, have shown sensitivity to cysteines present in sequencing polymerases. The cysteines normally form a disulfide bridge, however in the presence of sequencing solutions and conditions, the disulfide bridge may break to form two reactive thiols. These thiols may act to prematurely cleave the linker and/or reversible terminator, acting as a weak reducing agent, increasing asynchronous shifts in sequencing runs that are detrimental to sequencing accuracy. There is a need for a sequencing polymerase that has reduced interference with the modified nucleotides used in sequencing applications.

Disulfide bridges are highly conserved among thermophilic polymerases. Wildtype Thermococcus sp. 9° N-7 (9°N) shares about 80% homology with other family B archael polymerases, such as Pyrococcus furiosus (Pfu)), Pyrococcus horikoshii (Pho), Pyrococcus woesei (Pwo), and Pyrococcus abyssi (Pab). The structure and function relationships identifying key conserved amino acids among the family B DNA polymerases has been reported, for example in Gueguen et al. (Gueguen, Y., et al (2001), European Journal of Biochemistry, 268:5961-5969); Bergen, K., et al. (Bergen, K., et al. (2013), ChemBioChem, 14:1058-1062); each of which are incorporated by reference. Briefly, Gueguen et al. provides sequence alignments between a number of DNA polymerases and notes that the amino acid sequences of the DNA polymerases examined contains the six conserved motifs shared by the family B DNA polymerases and the three motifs for 3′->5′ exonuclease activity. Bergen provides crystal structures of two DNA polymerases, Thermococcus kodakaraerisis (KOD1) and Thermococcus sp. 9° N-7 (9°N), and demonstrates its close structural and functional similarities to other DNA polymerases of different families, such as KlenTaq. Structural data has implied that disulfides do not play a direct role in catalysis or substrate binding, but rather, it has been suggested that they contribute to enzyme thermostability. Studies assessing the removal of disulfides from family B archaeal polymerases have shown that the disulfides contribute to thermostability (Killelea T. and Connolly B A. ChemBioChem. 2011, 12:1330-36).

The applicants discovered the polymerases are capable of incorporating modified nucleotides at high temperatures, and advantageously do not degrade the nucleotides permitting longer sequencing read lengths and better accuracy. Provided herein are novel family B DNA polymerases wherein the conserved cysteines are mutated. As an initial test, the applicants mutated the cysteines at positions 429, 443, 507, and 510 to serine amino acids, as described in Table 7. Table 7 reports on the selective mutation of only C429S and C443S (disulfide bridge 1 (DB1)), only C507S and C510S (disulfide bridge 2 (DB2)); and all four cysteines C429S, C443S, C507S, and C510S (disulfide bridge 3 (DB3)). While serine was chosen as an initial mutation, any amino acid that eliminates the ability to form free thiols and does not perturb the stability nor function of the polymerase is envisioned (e.g., glycine, threonine, selenocysteine or alanine). Each of the variants lacking a cysteine was capable of incorporating modified nucleotides, and advantageously, the modified nucleotides exhibited greater stability (i.e., did not prematurely deblock or lose the detectable moiety) relative to a polymerase that contained one or more cysteines.

TABLE 7
Cysteine positions in this table are mutations relative
to the wild type P. horikoshii (SEQ ID NO: 1).

| Internal Ref # | Amino acids |
|---|---|
| DB-1 | C429S; C443S |
| DB-2 | C507S; C510S |
| DB-3 | C429S; C443S; C507S; C510S |

SGFSFOm129ac
(SEQ ID NO: 1)
MILDADYITEDGKPIIRIFKKENGEFKVEYDRNFRPYIYALLRDDSAIDEIKKITAQRHGKVVR
IVETEKIQRKFLGRPIEVWKLYLEHPQDQPAIRDKIREHPAVVDIFEYDIPFAKRYLIDKGLTP
AEGNEKLTFLAVAIAALYHEGEEFGKGPVIMISYADEEGAKVITWKKIDLPYVEVVSSEREMIK
RLIRVIKEKDPDVIITYNGDNFDFPYLLKRAEKLGIKLLLGRDNSEPKMQKMGDSLAVEIKGRI
HFDLFPVIRRTINLPTYTLEAVYEAIFGKPKEKVYADEIAKAWETGEGLERVAKYSMEDAKVTY
ELGREFFPMEAQLARLVGQPVWDVSRSSTGNLVEWELLRKAYERNELAPNKPDEKEYERRLRES
YEGGYVKEPEKGLWEGIVSLDFRSAGPSIIITHNVSPDTLNREGCEEYDVAPKVGHRFCKDEPG
FIPSLLGQLLEERQKIKKRMKESKDPVEKKLLDYRQRVIKILANSYYGYYGYAKARWYCKECAE
SVSAWGRQYIDLVRRELEARGFKVLYIDTDGLYATIPGVKDWEEVKRRALEFVDYINSKLPGVL
ELEYEGFYARGFFVTKKKYALIDEEGKIVTRGLEIVRRDWSEIAKETQARVLEAILKHGNVEEL
VKIVKDVTEKLTNYEVPPEKLVIYEQITRPINEYKAIGPHVAVAKRLMARGIKVKPGMVIGYIV
LRGDGPISKRAISIEEFDPRKHKYDAEYYIENQVLPAVERILKAFGYKREDLRWQKTKQVGLGA
WIKVKKS
SGFSONO_CFS
(SEQ ID NO: 19)
MILDADYITEDGKPIIRIFKKENGEFKVEYDRNFRPYIYALLRDDGAIDEIKKITAQRHGKVVR
IVETEKIQRKFLGRPIEVWKLYLEHPQDQPAIRDKIREHQAVVDIFEYDIPFAKRYLIDKGLTP
AEGNERLTFLAVAIAALYHEGEEFGKGPVIMISYADEEGAKVITWKKIDLPYVEVVSSEREMIK
RLIRVIKEKDPDVIITYNGDNFDFPYLLKRAEKLGIKLLLGRDDNEPKIQKMGDSLAVEIKGRF
HLDFLPVIRRTINLPTYTLEAVYKAFFGKPKDKVYADEIAKAWGTGEGLGRVAKYSMEDAKVTY
ELGREFFPMEAQLARLVGQPLWDVSRSSTGNLVEWFLLRKAYERNELAPNKPDEKEYERRLRES
YEGGYVKEPEKGLWEGIVSLDFRSAGPSIIIAHNVSPDTLNREGCEEYDVAPKLGHRFCKDEPG
FIPSLLGOLLEERQKIKKRMKESKDPVEKKPLDYRORVIKILANSFYGYYGYAKARWYCKECAE
SVSAWGRQYIDLVRRELEARGFKVLYIDTDGLYATIPGVKDWEEVKRRALEFVDYINSKLPGVL
ELEYGGFYARGLFVTKKKYALIDEEGKIVTRGLEIVRRDWSEIAKETQARVLEAILKHGNVEEL
VKIVKDVTEKLINYEVPPEKLVIYEQITRPINEYKAIGPHVAVAKRLMARGIKVKPGMVIGYIV
LRGDGPISKRAISIEEFDPRKHKYDAEYYIENQVLPAVERILKAFGYKREDLRWQKTKQVGLGA
WIKVKKSGGSGHHHHHH
SGFSONO_CFSnH
(SEQ ID NO: 20)
MILDADYITEDGKPIIRIFKKENGEFKVEYDRNFRPYIYALLRDDGAIDEIKKITAQRHGKVVR
IVETEKIQRKFLGRPIEVWKLYLEHPQDQPAIRDKIREHQAVVDIFEYDIPFAKRYLIDKGLTP
AEGNERLTFLAVAIAALYHEGEEFGKGPVIMISYADEEGAKVITWKKIDLPYVEVVSSEREMIK
RLIRVIKEKDPDVIITYNGDNFDFPYLLKRAEKLGIKLLLGRDDNEPKIQKMGDSLAVEIKGRF
HLDFLPVIRRTINLPTYTLEAVYKAFFGKPKDKVYADEIAKAWGTGEGLGRVAKYSMEDAKVTY
ELGREFFPMEAQLARLVGQPLWDVSRSSTGNLVEWELLRKAYERNELAPNKPDEKEYERRLRES
YEGGYVKEPEKGLWEGIVSLDFRSAGPSIIIAHNVSPDTLNREGCEEYDVAPKLGHRFCKDFPG
FIPSLLGQLLEERQKIKKRMKESKDPVEKKPLDYRORVIKILANSFYGYYGYAKARWYCKECAE
SVSAWGRQYIDLVRRELEARGFKVLYIDTDGLYATIPGVKDWEEVKRRALEFVDYINSKLPGVL
ELEYGGFYARGLFVTKKKYALIDEEGKIVTRGLEIVRRDWSEIAKETOARVLEAILKHGNVEEL
VKIVKDVTEKLTNYEVPPEKLVIYEQITRPINEYKAIGPHVAVAKRLMARGIKVKPGMVIGYIV
LRGDGPISKRAISIEEFDPRKHKYDAEYYIENQVLPAVERILKAFGYKREDLRWQKTKQVGLGA
WIKVKKS
SGFSFO_CFSnH
(SEQ ID NO: 21)
MILDADYITEDGKPIIRIFKKENGEFKVEYDRNFRPYIYALLRDDGAIDEIKKITAQRHGKVVR
IVETEKIQRKFLGRPIEVWKLYLEHPQDQPAIRDKIREHQAVVDIFEYDIPFAKRYLIDKGLTP
AEGNERLTFLAVAIAALYHEGEEFGKGPVIMISYADEEGAKVITWKKIDLPYVEVVSSEREMIK
RLIRVIKEKDPDVIITYNGDNFDFPYLLKRAEKLGIKLLLGRDDNEPKIQKMGDSLAVEIKGRF
HLDFLPVIRRTINLPTYTLEAVYKAFFGKPKDKVYADEIAKAWGTGEGLGRVAKYSMEDAKVTY
ELGREFFPMEAQLARLVGQPLWDVSRSSTGNLVEWELLRKAYERNELAPNKPDEKEYERRLRES
YEGGYVKEPEKGLWEGIVSLDERSAGPSIIIAHNVSPDTLNREGSEEYDVAPKLGHRFSKDEPG
FIPSLLGQLLEERQKIKKRMKESKDPVEKKPLDYRQRVIKILANSFYGYYGYAKARWYSKESAE
SVSAWGRQYIDLVRRELEARGFKVLYIDTDGLYATIPGVKDWEEVKRRALEFVDYINSKLPGVL
ELEYGGFYARGLFVTKKKYALIDEEGKIVTRGLEIVRRDWSEIAKETQARVLEAILKHGNVEEL
VKIVKDVTEKLTNYEVPPEKLVIYEQITRPINEYKAIGPHVAVAKRLMARGIKVKPGMVIGYIV
LRGDGPISKRAISIEEFDPRKHKYDAEYYIENQVLPAVERILKAFGYKREDLRWQKTKQVGLGA
WIKVKKS
SGFSFO_CFS
(SEQ ID NO: 22)
MILDADYITEDGKPIIRIFKKENGEFKVEYDRNFRPYIYALLRDDGAIDEIKKITAQRHGKVVR
IVETEKIQRKFLGRPIEVWKLYLEHPQDQPAIRDKIREHQAVVDIFEYDIPFAKRYLIDKGLTP
AEGNERLTFLAVAIAALYHEGEEFGKGPVIMISYADEEGAKVITWKKIDLPYVEVVSSEREMIK
RLIRVIKEKDPDVIITYNGDNEDFPYLLKRAEKLGIKLLLGRDDNEPKIQKMGDSLAVEIKGRE
HLDFLPVIRRTINLPTYTLEAVYKAFFGKPKDKVYADEIAKAWGTGEGLGRVAKYSMEDAKVTY
ELGREFFPMEAQLARLVGQPLWDVSRSSTGNLVEWELLRKAYERNELAPNKPDEKEYERRLRES
YEGGYVKEPEKGLWEGIVSLDERSAGPSIIIAHNVSPDTLNREGSEEYDVAPKLGHRFSKDFPG
FIPSLLGOLLEERQKIKKRMKESKDPVEKKPLDYRQRVIKILANSFYGYYGYAKARWYSKESAE
SVSAWGRQYIDLVRRELEARGFKVLYIDTDGLYATIPGVKDWEEVKRRALEFVDYINSKLPGVL
ELEYGGFYARGLFVTKKKYALIDEEGKIVTRGLEIVRRDWSEIAKETQARVLEAILKHGNVEEL
VKIVKDVTEKLINYEVPPEKLVIYEQITRPINEYKAIGPHVAVAKRLMARGIKVKPGMVIGYIV
LRGDGPISKRAISIEEFDPRKHKYDAEYYIENQVLPAVERILKAFGYKREDLRWQKTKQVGLGA
WIKVKKSGGSGHHHHHH

What is claimed is:

1. A polymerase comprising an amino acid sequence that is at least 80% identical to a continuous 500 amino acid sequence within SEQ ID NO: 1; comprising a mutation at amino acid position 306 or an amino acid position corresponding to position 306, wherein the mutation at amino acid position 306 comprises aspartic acid, glutamine, asparagine, alanine, serine, proline, valine, or glycine.

2. The polymerase of claim 1, wherein the mutation at amino acid position 306 is glycine, alanine, or valine.

3. The polymerase of claim 1, comprising leucine, isoleucine, valine, alanine, or glycine at amino acid position 341 or an amino acid position corresponding to position 341.

4. The polymerase of claim 1, comprising leucine, isoleucine, alanine, or glycine at amino acid position 341 or an amino acid position corresponding to position 341.

5. The polymerase of claim 1, comprising tyrosine, phenylalanine, tryptophan, leucine, isoleucine, or valine at amino acid position 494 or an amino acid position corresponding to position 494.

6. The polymerase of claim 1, comprising phenylalanine, tryptophan, leucine, isoleucine, or valine at amino acid position 494 or an amino acid position corresponding to position 494.

7. The polymerase of claim 1, comprising phenylalanine at amino acid position 494 or an amino acid position corresponding to position 494.

8. The polymerase of claim 1, comprising glutamic acid, aspartic acid, glutamine, asparagine, alanine, serine, proline, valine, or glycine at amino acid position 581 or an amino acid position corresponding to position 581.

9. The polymerase of claim 1, comprising tyrosine, phenylalanine, tryptophan, leucine, isoleucine, or valine at amino acid position 588 or an amino acid position corresponding to position 588.

10. The polymerase of claim 1, comprising lysine, arginine, histidine, glutamic acid, aspartic acid, glutamine, asparagine, alanine, serine, proline, valine, or glycine at amino acid position 280 or an amino acid position corresponding to position 280.

11. The polymerase of claim 1, comprising methionine, alanine, serine, leucine, isoleucine, valine, or cysteine at amino acid position 241 or an amino acid position corresponding to position 241.

12. The polymerase of claim 1, comprising asparagine, lysine, aspartic acid, glutamine, serine, threonine, tyrosine, or glutamic acid at amino acid position 236 or an amino acid position corresponding to position 236.

13. The polymerase of claim 1, comprising E306G, V341L, Y494F, E581G, and F588L.

14. The polymerase of claim 1, further comprising E280K, M241I, and N236D.

15. The polymerase of claim 1, further comprising a mutation at amino acid position 409 or an amino acid position corresponding to position 409.

16. The polymerase of claim 15, wherein the mutation at amino acid position 409 or the amino acid position corresponding to position 409 is alanine, glutamine, tyrosine, phenylalanine, isoleucine, valine, cysteine, serine, or histidine.

17. The polymerase of claim 1, comprising:
an alanine or serine at amino acid position 409 or the amino acid position corresponding to position 409; a glycine at amino acid position 410 or an amino acid position corresponding to position 410; and a proline, valine, glycine, isoleucine, or serine at amino acid position 411 or an amino acid position corresponding to position 411.

18. The polymerase of claim 1, further comprising at least one of the following:
a serine at amino acid position 429 or an amino acid position corresponding to position 429; a serine at amino acid position 443 or an amino acid position corresponding to position 443; a serine at amino acid position 507 or an amino acid position corresponding to position 507; and a serine at amino acid position 510 or an amino acid position corresponding to position 510.

19. A method of incorporating a modified nucleotide into a nucleic acid sequence comprising combining in a reaction vessel: (i) a nucleic acid template, (ii) a nucleotide solution, and (iii) a polymerase, wherein the polymerase is a polymerase of claim 1.

20. A method of sequencing a nucleic acid sequence comprising:
a. hybridizing a nucleic acid template with a primer to form a primer-template hybridization complex;
b. contacting the primer-template hybridization complex with a DNA polymerase and modified nucleotides, wherein the DNA polymerase is the polymerase of claim 1, wherein the modified nucleotide comprises a detectable label;
c. incorporating a modified nucleotide into the primer-template hybridization complex with the DNA polymerase to form a modified primer-template hybridization complex; and
d. detecting the detectable label; thereby sequencing a nucleic acid sequence.
